(19) United States
(12) Patent Application Publication
(10) Pub. No.: US 2005/0042219 A1
(43) Pub. Date: Feb. 24, 2005

(54) Engineered Fab' fragment anti-tumor necrosis factor alpha in combination with disease modifying anti-rheumatic drugs

(76) Inventors: Susan L. Woulfe, Woodbury, MN (US); Rita Jain, Roslyn Harbor, NY (US); Aimee Burr, Chicago, IL (US)

Correspondence Address: Carol M. Nielsen;Winstead Sechrest & Minick P.C., P.O. Box 50784, Dallas, TX, 75201, US

(21) Appl. No.: 10/728,420

(22) Filed: Dec. 5, 2003

Related U.S. Application Data
(60) Provisional application No. 60/431,053, filed on Dec. 5, 2002.

(51) Int. Cl. A61K 39/395; A61K 31/525

(52) U.S. Cl. 424/145.1; 514/251

(57) ABSTRACT

The present invention provides a combination therapeutic composition useful in modulating, treating and/or preventing inflammatory diseases. The therapeutic composition includes antibody Fab′ fragment CDP870 that is specific for antigenic determinants of tumor necrosis factor alpha and at least one Disease Modifying Anti-Rheumatic Disease drug such as methotrexate or sulfasalazine. The therapeutic composition can be administered to either a human or animal subject in single or multiple doses over time in the treatment of rheumatoid arthritis, Crohn's Disease or any other inflammatory disease.

### CROSS-REFERENCE TO RELATED APPLICATIONS

The present application claims priority under Title 35, United States Code, § 119(e)(1) of U.S. Prov. Pat. App. Ser. No. 60/431,053, filed Dec. 5, 2002.

### REFERENCE TO A “SEQUENCE LISTING,” ON A COMPACT DISK

This application includes a sequence listing, pursuant to 37 CFR 1.821, contained on a compact disk, which is incorporated fully into this application by this reference.

The compact disk is labeled as follows:

Applicant: Woulfe, et al.
Title: Engineered Fab′ Fragment Anti-Tumor Necrosis Factor Alpha in Combination with Disease Modifying Anti-Rheumatic Drugs
Docket No.: 122294-1010
Creation Date: Dec. 3, 2003
Filing Date: Dec. 5, 2003

The compact disk contains the following file in ASCII file format:

| File Name | File size | Creation Date |
|---|---|---|
| Sequence.txt | 24 kb | Dec. 3, 2003 |

### FIELD OF INVENTION

This invention relates to the treatment of inflammatory disease in a subject by administration of a combination of a disease modifying anti-rheumatic drug with an anti-tumor necrosis factor antibody. More specifically, this invention pertains to the treatment of rheumatoid arthritis and other rheumatic-related diseases by a combination therapy including methotrexate, and/or sulfasalazine and an anti-tumor necrosis factor alpha Fab′ fragment antibody.

### BACKGROUND OF THE INVENTION

Tumor Necrosis Factor Alpha (TNFα) is a cytokine that is released by and interacts with cells of the immune system. TNFα is released by macrophages that have been activated by lipopolysaccharides of gram-negative bacteria. As such, TNFα appears to be an endogenous mediator of central importance involved in the development and pathogenesis of endotoxic shock associated with bacterial sepsis. As early as 1985, antibodies to TNFα were proposed for the prophylaxis and treatment of endotoxic shock (Beutler et al., Science, 234, 470-474, 1985). By 1988, Shimamoto et al. described the use of murine monoclonal antibodies against TNFα and their use in preventing endotoxic shock in mice (Immunology Letters, 17, 311-318, 1988). The use of anti-TNFα antibodies in the treatment of septic shock is further discussed in Kirschenbaum et al. (Critical Care Medicine, 26, 1625-1626, 1998).

In addition to endotoxic shock, TNFα has also been shown to contribute to a number of inflammatory human chronic diseases such as Crohn's disease, ulcerative colitis, and in particular, rheumatoid arthritis. In 1991, Kaffer et al. found that mice transgenic for human TNFα produce high levels of TNFα constitutively and develop a spontaneous, destructive polyarthritis resembling rheumatoid arthritis (Kaffer et al., EMBO J., 10, 4025 4031, 1991). Increased levels of TNFα can also be found in both the synovial fluid and peripheral blood of patients suffering from rheumatoid arthritis. TNFα is therefore referred to as a pro-inflammatory cytokine.

Rheumatoid arthritis is a disease that affects both the large and small joints, and if untreated can cause permanent joint damage. Initially, in order to reduce pain and inflammation associated with rheumatoid arthritis, aspirin or Non-Steroidal Anti-Inflammatory Drugs (NSAIDs), such as ibuprofen or naproxen, are used. However, prolonged use of these can cause stomach upset, ulceration, and bleeding from the stomach and intestines. A new class of drugs, called Cox-2 inhibitors (e.g. Celebrex), is also used to reduce pain and inflammation associated with arthritic joints but without the risk of injuring the stomach and intestines.

When these pain and inflammation reducing treatments fail, a second line of drugs, called Disease Modifying Anti-Rheumatic Drugs (DMARDs), are used. DMARDs are immunosuppressive drugs (or an “immunosuppressant”) that work by suppressing the immune's system attack on joints. DMARDs are considered remittive because they can slow down the disease process; however, use of DMARDs seldom leads to a complete remission. Because it may take 6 to 8 months for the DMARDs to evoke a response, they are viewed as slow-acting drugs. The DMARD of choice is methotrexate since it has shown long term effectiveness with less long-term toxicity as compared to other DMARDs such as hydroxychloroquine, D-penicillamine and injectable gold.

Antibodies have also been designed to treat rheumatoid arthritis by specifically acting on inflammatory cytokines. Initially, the antibodies that were studied were murine monoclonal antibodies (murine Mabs). However, these antibodies have limited utility as human therapeutics since they are themselves foreign antigens that cause antibodies to be produced against them once they are introduced in a human. This response, known as human anti-mouse response (HAMA), is enhanced with repeated exposure to the murine antibody. Therefore, Mabs are generally used as a human therapeutic only where repeated application is not required.

To make murine Mabs suitable for repeated human use in humans, the antibodies were “humanized.” Humanization of Mabs involved the creation of human chimeric antibodies having murine variable regions and human constant regions. However, these antibodies still contained significant proportions of non-human amino acid sequence (i.e. complete non-human variable domains where the antigen binding domains are located) and, if administered over a long period of time, elicit an HAMA response.

Efforts have been made to further reduce the murine component in the variable regions, and in particular in the essential antigen binding domains. In one approach, the several complementary determining regions (CDRs) of murine Mabs are grafted onto the framework regions of the variable domains of a human immunoglobulin. However, these antibodies generally have a reduced antigen binding activity when compared to antibodies from which the variable CDRs were derived. In another approach, a completely humanized monoclonal antibody was developed comprising human variable and human Fc constant regions. Although human antibodies are not antigenic per se in another human, “autoantibodies” to human antigens can be formed which can limit the efficacy of repeated antibody therapy.

Recently, two anti-TNFα biologics specific to TNF, called etanercept and infliximab, have received FDA approval for treating rheumatoid arthritis. Etanercept (FDA approval in 1998) is manufactured and marketed by Immunex Corporation as Enbrel. It is a recombinant fusion protein comprising two p75 soluble TNF-receptor domains linked to the Fc portion of a human immunoglobulin IgG1 and is produced by recombinant DNA technology in a Chinese hamster ovary mammalian cell expression system. Infliximab (FDA approval in 2001) is marketed by Centocor Corporation as Remicade. It is a chimeric antibody having murine anti-TNFα variable domains and human IgG1 Fc regions (constant regions). The chimeric antibody, Infliximab, has been used in combination with methotrexate to suppress the inflammatory response generated by this antibody.

Available anti-TNFα antibodies on the market and in development possess non-human variable domains and human constant regions. As a result, undesirable inflammatory responses may occur during treatment therapy and such response is a problem. For example, the human constant regions of IgG1 type of antibody activate proinflammatory responses through FcR-mediated mechanisms and/or by activation of the complement cascade.

To alleviate the problems associated with the inflammatory response, a novel and more effective antibody, CDP870 (an anti-TNFα Fab′ fragment), was designed without the need for the heavy constant regions. CDP870 uses a human variable domain framework where only the complementary domain regions are non-human. Initially, the effects of such Fab′ fragment were unknown. However, such fragment has been found to dramatically reduce an inflammatory response. To further reduce or completely eliminate the inflammatory response and also to reduce the immunosuppressive response generated by the Fab′ fragment, a new and improved method of treatment has been discovered.

### SUMMARY OF THE INVENTION

The present invention provides a method of treating, preventing and/or inhibiting an inflammatory disease or disorder by administering an anti-tumor necrosis factor alpha antibody Fab′ fragment and at least one disease modifying anti-rheumatic drug to a subject in therapeutically effective amounts. Types of inflammatory disease or disorder that can be treated and/or prevented include, but are not limited to, rheumatoid arthritis, Crohn's Disease, Graft Versus Host Disease (“GVHD”), as well as any other rheumatic-related disease. The preferred anti-tumor necrosis factor alpha antibody Fab′ fragment is CDP870, an antibody produced in E. coli that is modified by attachment at a cysteine site to cross-linked polyethylene glycol chains linked by a single maleimide group, and the disease modifying anti-rheumatic drug is methotrexate. In another embodiment, CDP870 is administered with the disease modifying anti-rheumatic drug sulfasalazine. In addition, CDP870 can be used in combination with both methotrexate and sulfasalazine to treat inflammatory diseases in the present invention. The subject invention also provides a composition and a kit for treatment, inhibition or prevention of an inflammatory disease.

### BRIEF DESCRIPTION OF THE DRAWINGS

For better understanding of the invention and to show by way of example how the invention may be carried into effect, reference is now made to the detail description of the invention along with the accompanying figures in which corresponding numerals in the different figures refer to corresponding parts and in which:

FIG. 1 depicts the amino acid sequence of the CDRs of hTNF40 (SEQ ID NOS: 1 to 7) where CDR H2′ is a hybrid CDR having C-terminal six amino acids from the H₂CDR sequence of a human subgroup 3 germline antibody and the amino acid changes to the sequence resulting from the hybridization are underlined.

FIG. 2 shows the nucleotide and predicted amino acid sequence of murine hTNF40V1 (SEQ ID NO: 99).

FIG. 3 shows the nucleotide and predicted amino acid sequence of the murine hTNF40Vh (SEQ ID NO: 100).

FIG. 4 shows the framework regions of the human light chain subgroup 1 compared to the framework regions of the hTNF40 light chain (SEQ ID NOS: 83-90).

FIG. 5 shows the framework regions of the human heavy chain subgroup 1 and subgroup 3 compared to the framework regions of the hTNF40 heavy chain (SEQ ID NOS: 91 to 98 and 106 to 109).

FIG. 6 depicts the structure of compound CDP870 comprising a modified Fab′ fragment derived from antibody hTNF40 covalently linked via a cysteine residue to a lysyl-maleimide linker wherein each amino group on the lysyl residue has covalently attached to it a methoxy PEG residue.

FIG. 7 shows the vector pTTO.

FIG. 8 shows vector pTTO-1.

FIG. 9 shows vector pTTQ9.

FIG. 10 shows the sequence of the OmpA oligonucleotide adapter (SEQ ID NO: 101). Internal restriction sites are shown in bold. The 5′ XhoI cohesive end ligates into the vector Sal1 site, blocking it. S.D. represents the OmpA Shine Dalgarno sequence;

FIG. 11 shows vector pACYC184.

FIG. 12 shows vector pTTO-2.

FIG. 13 shows vector pDNAbEng-G1.

FIG. 14 shows the oligonucleotide cassettes encoding different intergenic sequences for E. coli modified Fab expression (SEQ ID NOS: 102 to 105).

FIG. 15 shows the nucleotide and predicted amino acid sequence of gh3hTNF40.4 (SEQ ID NO: 11).

FIG. 16 shows the mean plasma concentrations of CDP870 during the 55 days after the administration of a single 400 mg dose.

FIG. 17 shows the mean dose-adjusted MTX plasma concentrations following administration of MTX alone were not significantly different than those observed after coadministration of MTX with a single dose (SD) of CDP870.

FIG. 18 shows the heavy chain mature protein sequence of CDP870 (SEQ ID NO: 115).

FIG. 19 shows the light chain mature protein sequence of CDP870 (SEQ ID NO: 113).

FIG. 20 shows the nucleic acid sequences of CDP870 as sense and anti-sense strands (SEQ ID NOS: 116 and 117).

### DETAILED DESCRIPTION OF THE INVENTION

The present invention provides a method for treating, preventing, and/or inhibiting a disease mediated by tumor necrosis factor alpha (TNFα) by administering at least one Disease Modifying Anti-Rheumatic Drug (DMARD) with an antibody Fab′ fragment having specificity for antigenic determinants of TNFα. In one preferred embodiment, the antibody Fab′ fragment is CDP870, an anti-TNFα antibody and the DMARD is methotrexate. In another preferred embodiment, the antibody Fab′ fragment is CDP870 and the DMARD is sulfasalazine. In addition, methotrexate and sulfasalazine can be combined to form the DMARD that is administered with CDP870 in the method of treatment of the present invention.

### Engineered Anti-Tumor Necrosis Factor Alpha Antibody

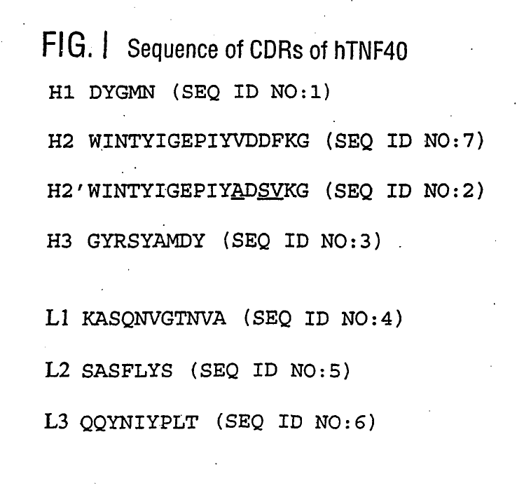

The present invention provides an antibody molecule having specificity for TNFα comprising a heavy chain having a variable domain comprising CDRs (as defined by Kabet et al. in Sequences of Proteins of Immunological Interest, US Department of Health and Human Services, NIH, USA, 1987) (hereafter Kabat) with the sequence given as H1 in FIG. 1 (SEQ ID NO: 1) for CDRH1, as H2′ or H2 in FIG. 1 (SEQ ID NO: 2 or SEQ ID NO: 7) for CDRH2 or as H3 in FIG. 1 (SEQ ID NO: 3) for CDRH3 and a light chain where the variable domain includes CDRs (as defined by Kabat) having the sequence given as LI in FIG. 1 (SEQ ID NO: 4) for CDRLI, as L2 in FIG. 1 (SEQ ID NO: 5) for CDRL2 or as L3 in FIG. 1 (SEQ ID NO: 6) for CDRL3.

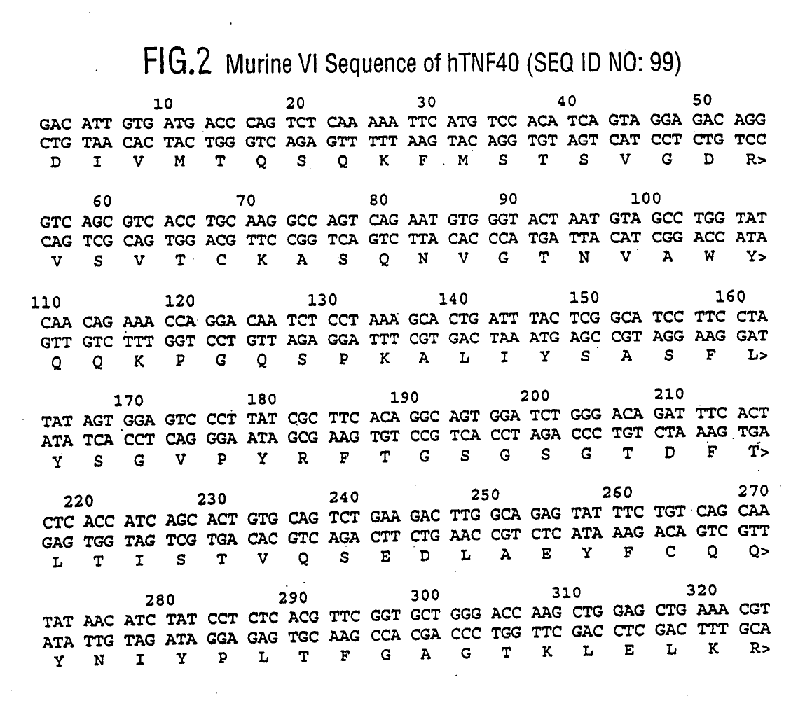

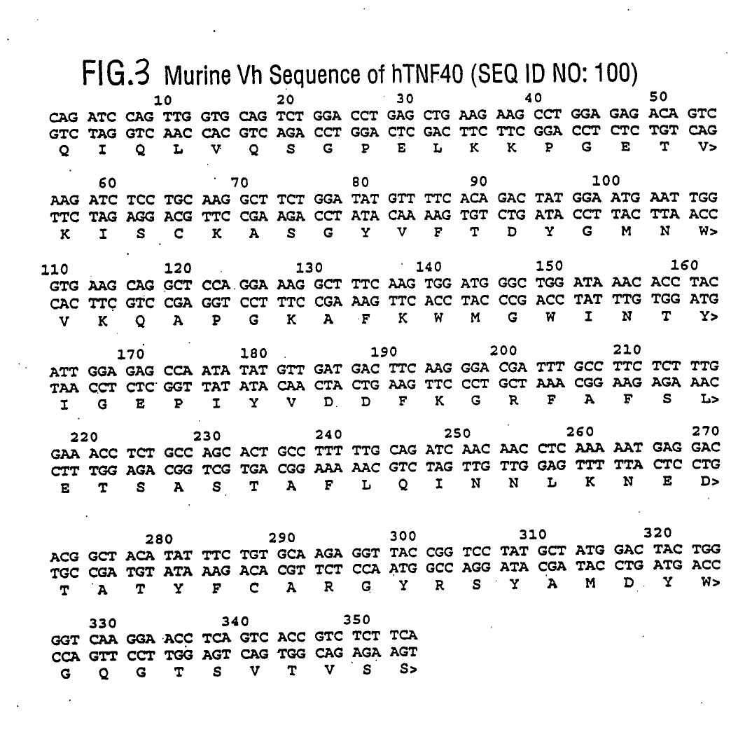

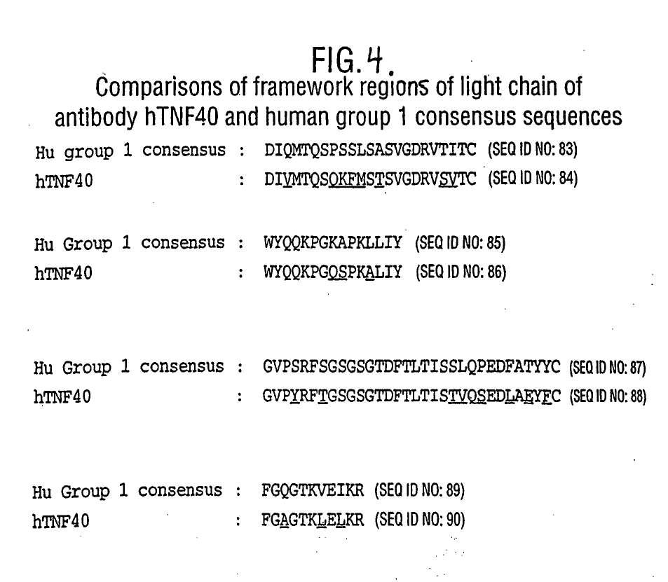

The CDRs given in SEQ IDS NOS: 1 and 3 to 7 in FIG. 1 are derived from a mouse monoclonal antibody hTNF40. However, SEQ ID NO: 2 consists of a hybrid CDR. The hybrid CDR comprises part of heavy chain CDR2 from mouse monoclonal antibody hTNF40 (SEQ ID NO: 7) and part of heavy chain CDR2 from a human group 3 germline V region sequence. The complete sequences of the variable domains of the mouse hTNF40 antibody are shown in FIG. 2 (light chain) (SEQ ID NO: 99) and FIG. 3 (heavy chain) (SEQ ID NO: 100).

The anti-TNFα antibody of the present invention is a CDR-grafted antibody molecule. Construction of CDR-grafted anti-TNFα antibodies of the present invention are described in WO 98/25971 and WO 01/94585, the entire contents of which are incorporated herein by reference. The term “a CDR-grafted antibody molecule” refers to an antibody molecule where the heavy and/or light chain contains one or more CDRs from a donor antibody grafted into a heavy and/or light chain variable region framework of an acceptor antibody. In comparison, a chimeric antibody is an antibody having unmodified non-human variable domains fused to the constant regions of the light and heavy chains of a human antibody. A humanized antibody is an antibody that includes not only the fused variable domains (usually murine) to human constant regions, but also altered selected variable domain framework residues that more closely match the most related available human variable template sequence. A CDR grafted antibody is an antibody having non-human CDR domains from an antibody grafted into the most closely related human antibody sequence available so that only the CDR domains are non-human in origin. CDR grafting antibodies have reduced immunogenicity and therefore also a reduced human-anti-mouse (HAMA) immune response.

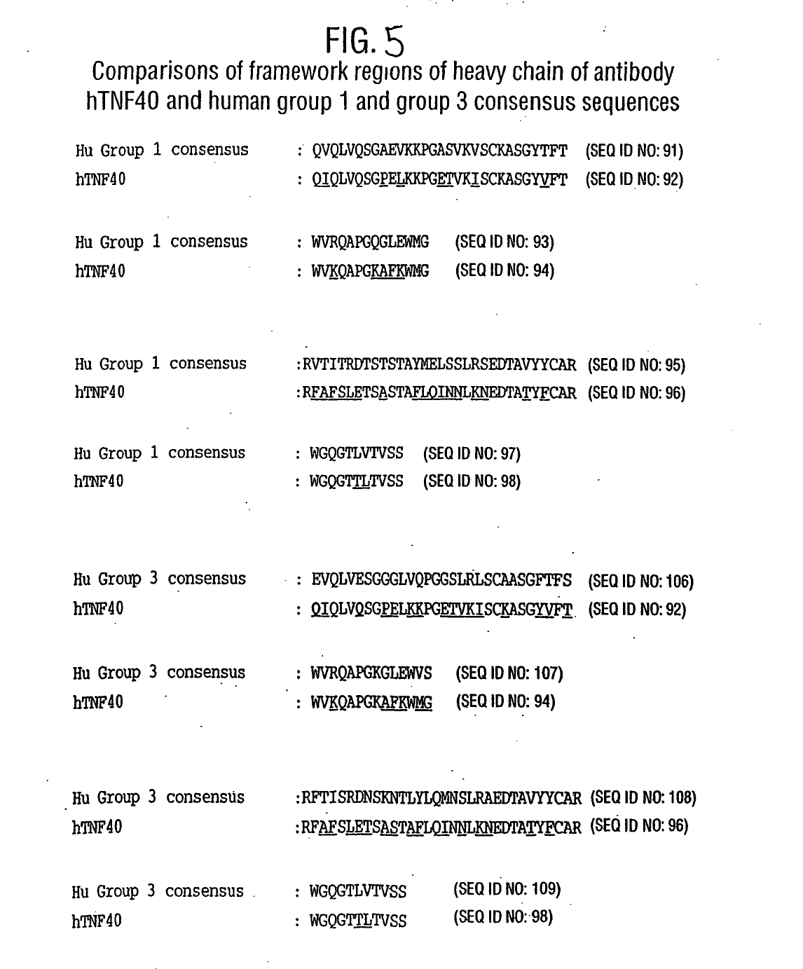

Any appropriate acceptor variable region framework sequence may be used having regard to the class/type of the donor antibody from which the CDRs are derived including mouse, primate and human framework regions. Preferably, the CDR-grafted antibody has a human acceptor framework region. Examples of human framework regions that can be used in the present invention are KOL, NEWM, REI, EU, TUR, TEI, LAY and POM (Kabat, supra). For example, KOL and NEWM can be used for the heavy chain, REI can be used for the light chain and EU, LAY and POM can be used for both the heavy chain and the light chain. The preferred framework regions for the light chain are the human group 1 framework regions shown in FIG. 4 (SEQ ID NOS: 83, 85, 87 and 89). The preferred framework regions for the heavy chain are the human group 1 and group 3 framework regions shown in FIG. 5 (SEQ ID NOS: 91, 93, 95 and 97 and SEQ ID NOS: 106, 107, 108 and 109), respectively.

It is preferred to use as the acceptor antibody one having chains which are homologous to the chains of the donor antibody. The acceptor heavy and light chains do not necessarily need to be derived from the same antibody and may, if desired, comprise composite chains having framework regions derived from different chains.

Also, the framework regions do not have to have the exact same sequence as those of the acceptor antibody. For instance, unusual residues may be changed to the more frequently occurring residues for that acceptor chain class or type. Alternatively, selected residues in the acceptor framework regions may be changed so that they correspond to the residue found at the same position in the donor antibody. Such changes should be kept to the minimum necessary to recover the affinity of the donor antibody.

If the acceptor heavy chain has human group 1 framework regions as shown in FIG. 5 (SEQ ID NOS: 91, 93, 95 and 97), then the acceptor framework regions of the heavy chain comprise, in addition to one or more donor CDRs, donor residues at positions 28, 69 and 71 (according to Kabat). As used herein, the donor residues are residues from the donor antibody, i.e. the antibody from which the CDRs were originally derived.

Alternatively, if the acceptor heavy chain has human group 1 framework regions, then the acceptor framework regions of the heavy chain comprise, in addition to one or more donor CDRs, donor residues at positions 28, 38, 46, 67, 69 and 71 (according to Kabat).

If the acceptor heavy chain has human group 3 framework regions as shown in FIG. 5 (SEQ ID NOS: 106, 107, 108 and 109), then the acceptor framework regions of the heavy chain comprise, in addition to one or more donor CDRs, donor residues at positions 27, 28, 30, 48, 49, 69, 71, 73, 76 and 78 (according to Kabat).

If the acceptor light chain has human group 1 framework regions as shown in FIG. 4 (SEQ ID NOS: 83, 85, 87 and 89) then the acceptor framework regions of the light chain comprise donor residues at positions 46 and 60 (according to Kabat).

The anti-TNFα antibody molecule used in the present invention is preferably a Fab′ fragment. The antibody molecule of the present invention may also be a Fab fragment, a (Fab′)₂ fragment, or an Fᵥ fragment. By definition, a Fab′ fragment is a Fab fragment that has additional cysteine residues at the carboxy terminus of CH1 and one or more cysteines from the antibody hinge region. A (Fab′)₂ fragment is two Fab′ fragments paired between hinge cysteines that allow the antibody molecule to contain two antigen binding sites. A Fab fragment means that portion of the antibody molecule that contains the sites for binding antigens including the first constant heavy (CH1) and constant light (CL) chains and the variable regions of the heavy chain (VH) and the light chain (VL). A Fᵥ fragment is that portion of an antibody that is responsible for the antigen specificity consisting of VH and VL.

DNA sequences that encode the antibody Fab, Fab′2, and Fv fragments of the present invention can be obtained by methods well known to those skilled in the art. For example, DNA sequences coding for part or all of the antibody heavy and light chains may be synthesized as desired from the determined DNA sequences or on the basis of the corresponding amino acid sequences. Standard techniques of molecular biology may be used to prepare DNA sequences coding for the antibody molecule of the present invention. Desired DNA sequences may be synthesized completely or in part using oligonucleotide synthesis techniques. Site-directed mutagenesis and polymerase chain reaction (PCR) techniques may be used as appropriate.

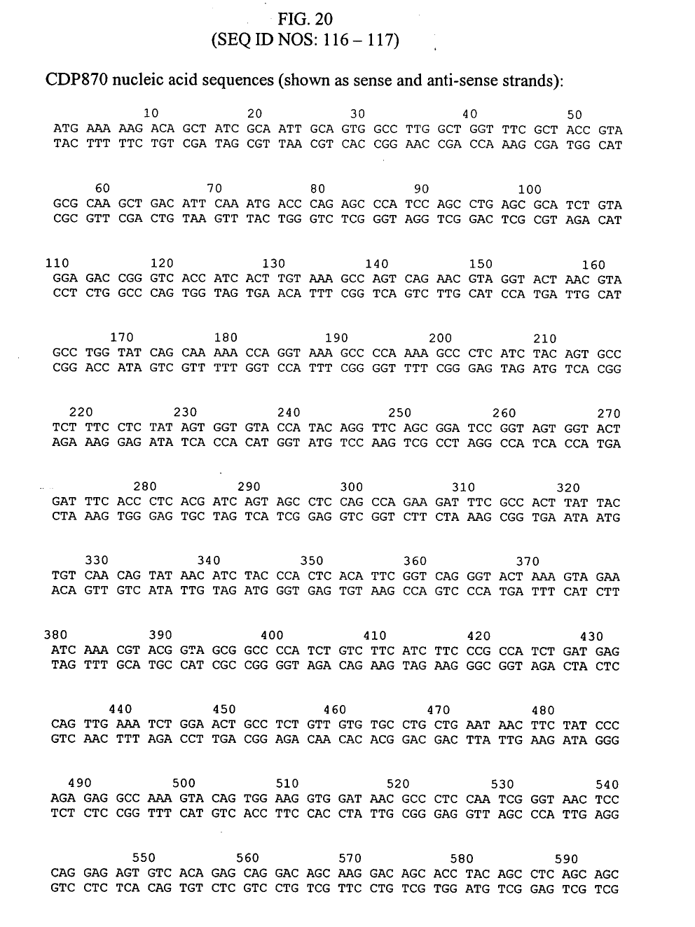

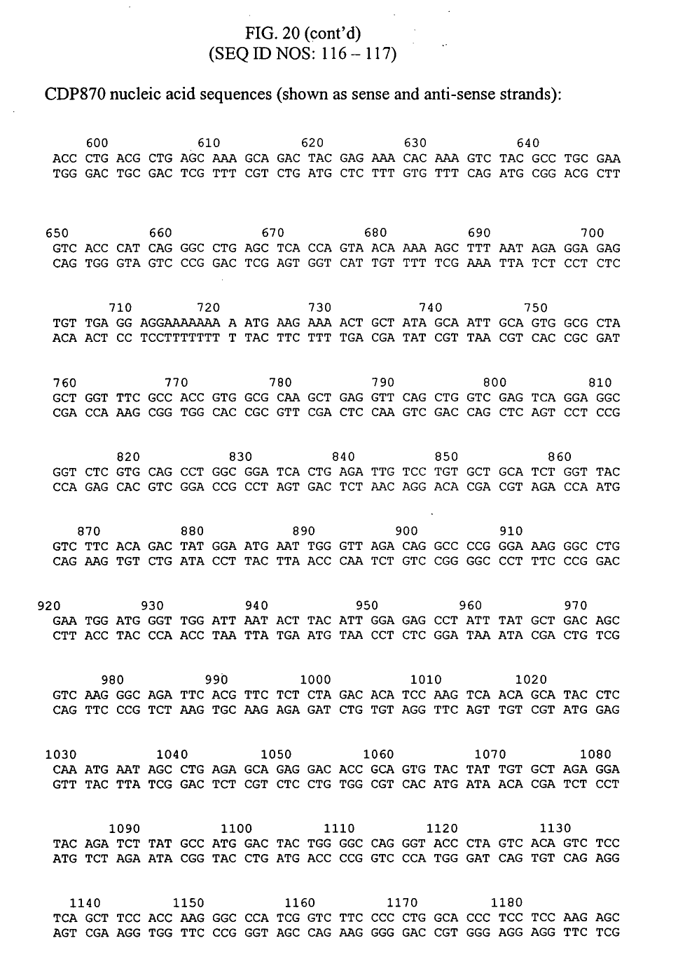

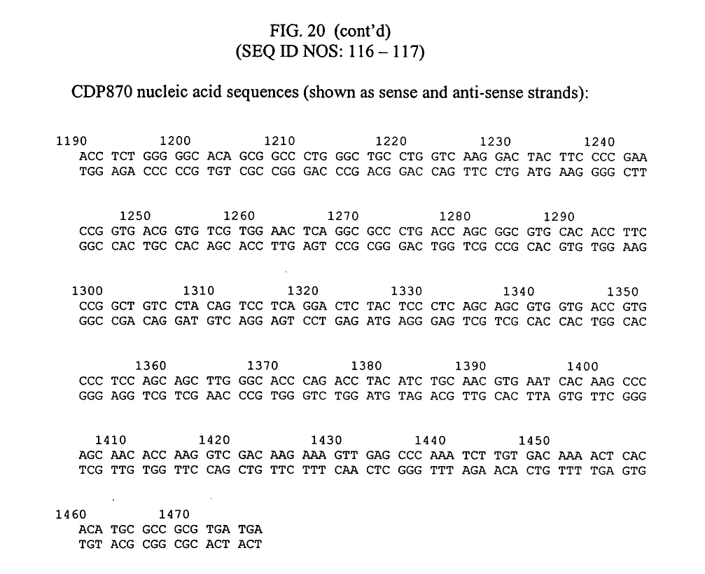

Preferably, the anti-TNFα antibody molecule used in the present invention is a Fab′ fragment antibody having a modification of the addition of one or more amino acids to the C-terminal end of its heavy chain to allow for the attachment of an effector molecule. The additional amino acids form a modified hinge region containing one or two cysteine residues that allow the effector molecule to attach. The antibody Fab′ fragment has a heavy chain with the protein (amino acid) sequence given as SEQ ID NO: 115 and the light chain having its protein (amino acid) sequence given as SEQ ID NO: 113 as shown in FIGS. 18 and 19. The coding sequences for CDP870 nucleic acid sequences, shown as sense and anti-sense strands, are depicted in FIG. 20 (SEQ ID NOS: 116 and 117).

The antibody Fab′ fragment may have one or more effector molecules attached to it. The effector molecules may be attached to the fragment through any available amino acid side-chain or terminal amino acid functional group located in the fragment, for example any free amino, imino, hydroxyl or carboxyl group. The effector group used in the present invention is generally a polymer molecule that is attached to the fragment to increase its half-life in vivo. The polymer molecule can be either a synthetic polymer (e.g. an optionally substituted straight or branched chain polyalkylene or polyoxyalkylene polymer) or a naturally occurring polymer (e.g. a branched or unbranched polysaccharide such as a homo- or hetero-polysaccharide).

Particular optional substituents that may be present on the above-mentioned synthetic polymers include one or more hydroxy, methyl or methoxy groups. Examples of synthetic polymers include optionally substituted straight or branched chain polyethylene glycol, polypropylene glycol, polyvinylalcohol or derivatives thereof; especially preferred is an optionally substituted polyethylene glycol such as methoxypolyethylene glycol or derivatives thereof. Naturally occurring polymers can include lactose, amylose, dextran, glyccogen or derivatives thereof. “Derivatives” include reactive derivatives, for example thiol-selective reactive groups such as maleimides and the like. The reactive group may be linked either directly to or through a linker segment to the polymer. The residue of such a group will in some instances form part of the product as the linking group between the antibody fragment and the polymer.

The size of the polymer may be varied as desired, but is generally in an average molecular weight range from 500 Da to 50000 Da, preferably from 5000 to 40000 Da and more preferably from 25000 to 40000 Da. The polymer size can also be selected on the basis of the intended use of the product. For example, if the final product is intended to leave the circulation and penetrate tissue, for example for use in the treatment of a tumor, it's advantageous to use a small molecular weight polymer (e.g. a molecular weight of around 5000 Da). For applications where the product remains in the circulation, it may be advantageous to use a higher molecular weight polymer (e.g. a molecular weight in the range from 25000 Da to 40000 Da).

Preferred polymers attached to the antibody Fab′ fragment, also referred to herein as “Fab fragment”, of the present invention include a polyalkylene polymer such as a polyethylene glycol, or a methoxypolyethylene glycol having a molecular weight in the range from about 20000 Da to about 40000 Da. Each polymer molecule attached to the antibody Fab′ fragment is covalently linked to the sulphur atom of a cysteine residue located in the antibody Fab′ fragment. The covalent linkage can be a disulphide bond or a sulphur-carbon bond.

An activated polymer may be used as the starting material in the preparation of polymer-modified Fab′ fragments. The activated polymer can be any polymer containing a thiol reactive group such as an imide (e.g. maleimide), an alpha-halocarboxylic acid or ester (e.g. iodoacetamide), or a vinyl sulphone or a disulphide. Such starting materials may be obtained commercially (for example from Shearwater Polymers Inc., Huntsville, Ala., USA) or may be prepared from commercially available starting materials using conventional chemical procedures.

The preferred antibody Fab′ fragment of the present invention has polyethylene glycol (PEG) covalently attached according to the method disclosed in EP-A-0948544. With regards to attaching polyethylene glycol (PEG) moieties, reference is made to “Polyethylene Gycol Chemistry, Biotechnical and Biomedical Applications”, 1992, J. Milton Harris (ed), Plenum Press, New York, “Polyethylene Glycol Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York.

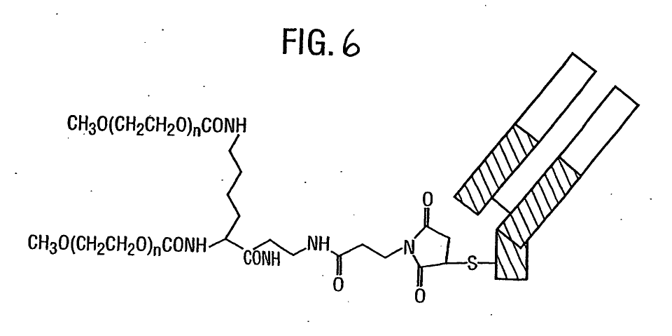

As depicted in FIG. 6, the preferred antibody Fab′ fragment has a maleimide group covalently linked to a single thiol group in a modified hinge region. A lysine residue is covalently linked to the maleimide group. To each of the amine groups on the lysine residue is attached a methoxypolyethylene glycol polymer having a molecular weight of approximately 20,000 Da. The total molecular weight of the entire effector molecule is therefore approximately 40,000 Da.

The preferred antibody Fab′ fragment anti-tumor necrosis factor alpha antibody of the present invention depicted in FIG. 6 is called CDP870. CDP870 is a novel antibody Fab′ fragment that has a high affinity for human TNFα, Kd of about 1.32×10⁻¹⁰M, and yet it does not neutralize TNFβ (lymphotoxin), a cytokine produced predominantly by T-cells that displays similar biological activities as TNFα but is often less potent. CDP870 is preferably produced in E. coli. The purified Fab′ fragment intermediate is then subsequently modified by attachment at a cysteine site to two-cross linked polyethylene glycol (PEG) chains bringing the total molecular weight to about 90000 Da. The cysteine site-PEG chains are linked by a single maleimide group to provide a prolonged half-life of about 11 days. By utilizing a specific single hinge thiol site for conjugation, the antibody of the present invention retains antigen binding and in vivo activities of the original antibody and has IgG-like pharmacokinetic profiles.

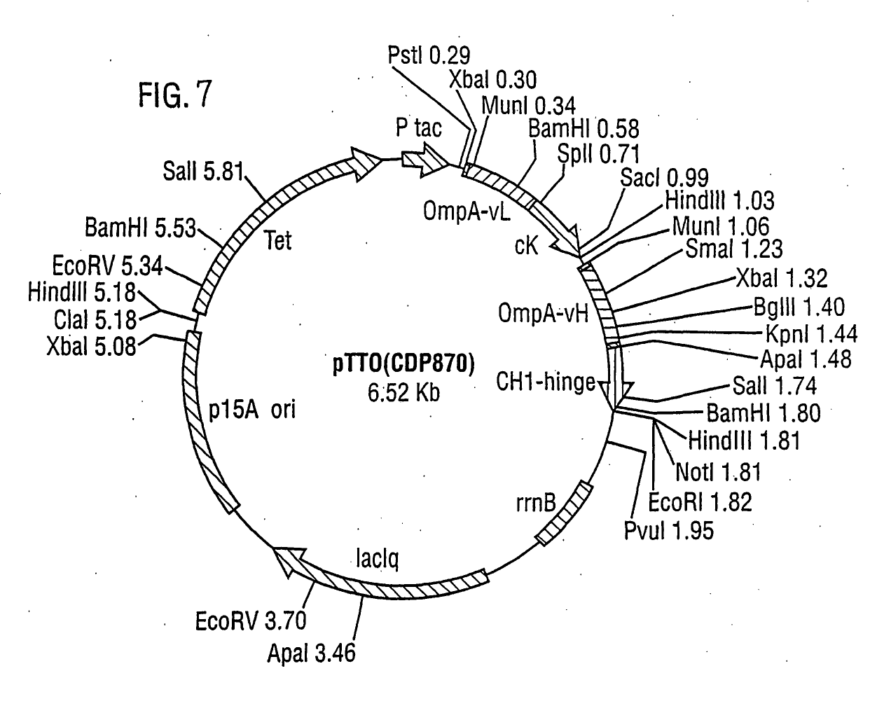

A preferred E. coli expression vector used in the present invention is the expression vector pTTO as shown schematically in FIG. 7. General methods by which the vector may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

DNA sequences that encode the antibody molecule of the present invention can be obtained by methods well known to those skilled in the art. For example, DNA sequences coding for part or all of the antibody heavy and light chains may be synthesized as desired from the determined DNA sequences or on the basis of the corresponding amino acid sequences.

DNA coding for acceptor framework sequences is widely available to those skilled in the art and can be readily synthesized on the basis of their known amino acid sequences.

Standard techniques of molecular biology may be used to prepare DNA sequences coding for the antibody molecule of the present invention. Desired DNA sequences may be synthesized completely or in part using oligonucleotide synthesis techniques. Site-directed mutagenesis and polymerase chain reaction (PCR) techniques may be used as appropriate.

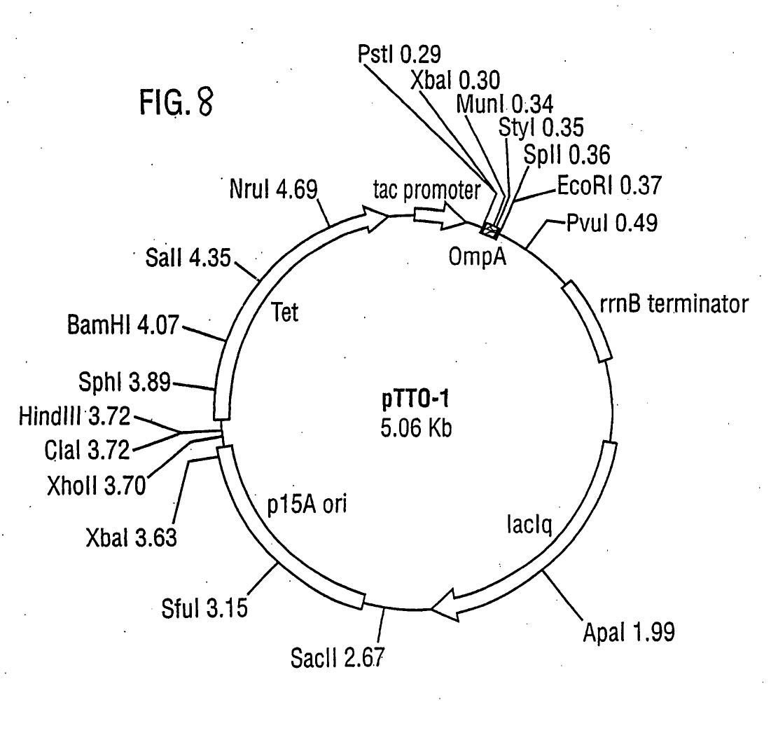

CDP870 may be constructed using the E. coli vector pTTO-1 shown in FIG. 8. The variable regions of antibody hTNF40 are sub-cloned into this vector and the intergenic sequence optimized to create pTTO (CDP870). The pTTO expression vector is designed to give rise to soluble, periplasmic accumulation of recombinant proteins in E. coli. The main features of this plasmid are:
(i) tetracycline resistance marker—antibiotic not inactivated by the product of resistance gene, hence selection for plasmid-containing cells is maintained;
(ii) low copy number—origin of replication derived from plasmid p15A, which is compatible with plasmids containing colE1 derived replicons;
(iii) strong, inducible tac promoter for transcription of cloned gene(s);
(iv) lacIq gene—gives constitutive expression of the lac repressor protein, maintaining the tac promoter in the repressed state until induction with IPTG/allolactose;
(v) OmpA signal sequence—gives periplasmic secretion of cloned gene(s) and translational coupling of OmpA signal sequence to a short lacZ peptide; and
(vi) giving efficient initiation of translation.

The vector has been developed for expression of modified Fab′ fragments from a dicistronic message by the design of a method to select empirically the optimum intergenic sequence from a series of four purpose-built cassettes.

Standard DNA techniques can be used in the construction of CDP870 including DNA restriction, agarose gel electrophoresis, ligation and transformation. Restriction enzymes and DNA modifying enzymes can be obtained from New England Biolabs or Boehringer Mannheim, and used according to the supplier's recommendations. DNA fragments can be purified from agarose using the GeneClean protocol (BIO 101). Oligonucleotides can be supplied by Oswel Oligonucleotide Service and synthesized at the 40 nm scale. Plasmid DNA can be isolated using Plasmid DNA Mini/Midi kits from Qiagen. PCR can be performed using Perkin Elmer ‘Amplitaq’ as recommended. DNA sequencing can be performed using the Applied Biosystems Taq cycle sequencing kit.

CDP870 may be prepared by expression in E. coli using the commonly available W3110 strain as the host. W3110 cultures can be grown in L-broth supplemented with tetracycline. For inductions, fresh overnight cultures (grown at 30° C.) can be diluted to OD₆₀₀ of 0.1 into 200 ml L-broth in a baffled flask and grown at 30° C. in an orbital incubator. At OD₆₀₀ of 0.5, IPTG can be added to 200 μM and samples (normalized for OD) taken at various intervals.

Periplasmic extraction can be performed by culturing the above samples then harvesting the cells by centrifugation. Following resuspension in extraction buffer (100 mM Tris-HCl, 10 mM EDTA, pH 7.4) samples can be incubated overnight at 30° C., then clarified by centrifugation.

Modified Fab concentrations can then be determined by ELISA. Plates can be coated at 4° C. overnight with anti-human Fd 6045 (2 μg/ml in coating buffer, physiological saline, 100 μl per well). After washing, 100 μl samples can be loaded per well with purified A5B7 gamma-1 Fab′, initially at 2 μg/ml, used as a standard. Samples can then be serially diluted 2-fold across the plate in sample conjugate buffer (per liter: 6.05 g trisaminomethane; 2.92 g NaCl; 0.1 ml Tween-20; 1 ml casein (0.2%)); plates can then be incubated for 1 hour at room temperature, with agitation. After washing and drying the plates, 100 μl of anti-human C-kappa (GD12)-peroxidase can be added (diluted in sample conjugate buffer). Incubation can be carried out at room temperature for 1 hour with agitation. After washing and drying the plates, 100 μl of substrate solution is added (10 ml sodium acetate/citrate solution (0.1 M pH 6); 100 μl H₂O₂ solution; 100 μl tetramethylbenzidine solution (10 mg/ml in dimethylsulphoxide)) and absorbance read at 630 nm four to six minutes after substrate addition.

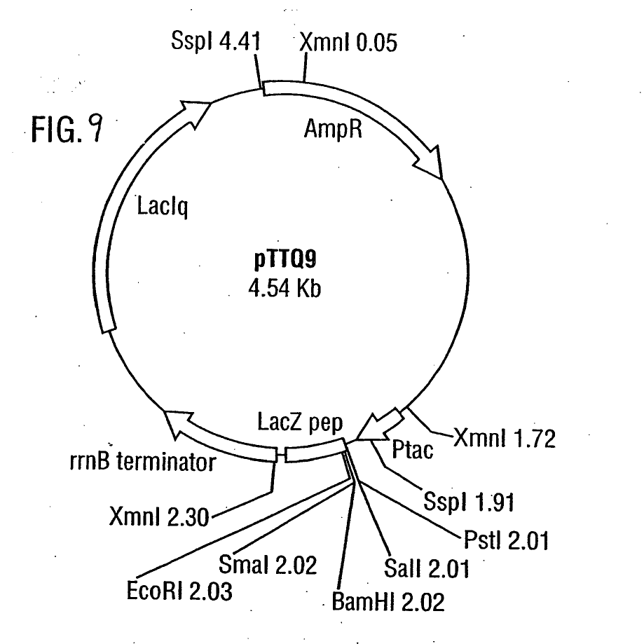

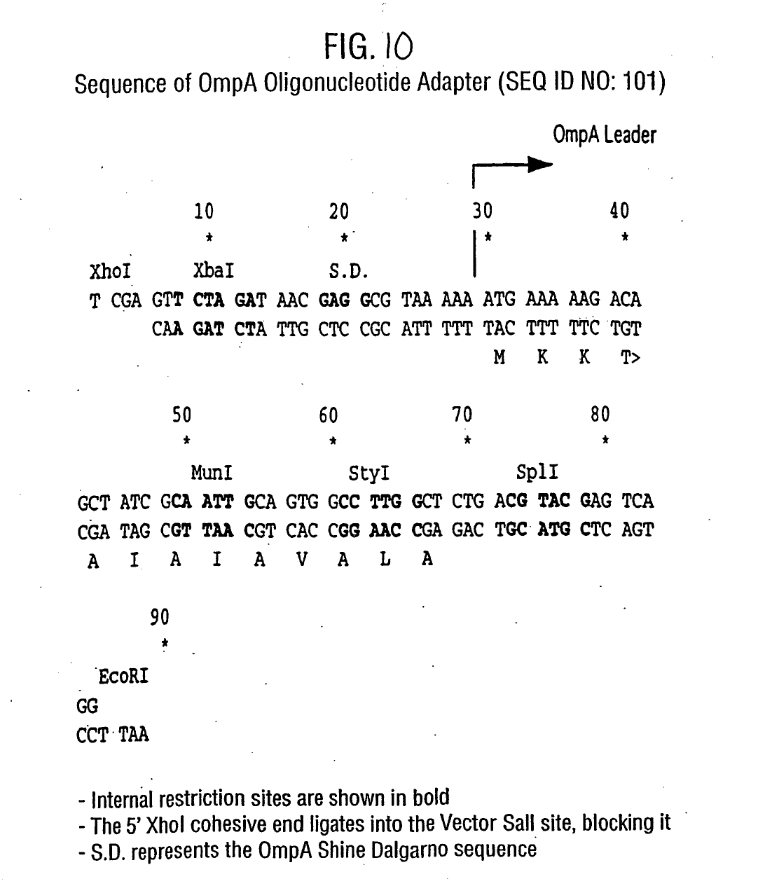

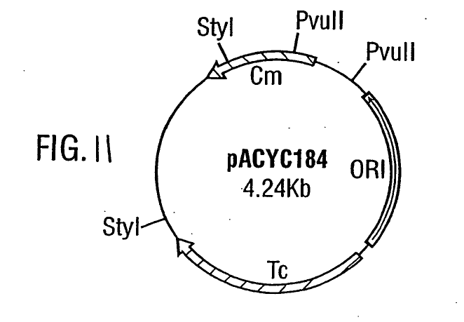

Construction of the plasmid pTTO-1 includes replacement of the pTTQ9 polylinker. Plasmid pTTQ9 can be obtained from Amersham and is shown in FIG. 9. An aliquot (2 μg) is digested with restriction enzymes SalI and EcoRI and the large DNA fragment (4520 bp) purified. Two oligonucleotides can be synthesized which, when annealed together, encode the OmpA polylinker region shown in FIG. 10. This sequence has cohesive ends that are compatible with the SalI and EcoRI ends generated by restriction of pTTQ9. By cloning this oligonucleotide ‘cassette’ into the pTTQ9 vector, the SalI site is not regenerated, but the EcoRI site is maintained. The cassette encodes the first 13 amino acids of the signal sequence of the E. coli outer-membrane protein Omp-A, preceded by the Shine Dalgamo ribosome binding site of the OmpA gene. In addition restriction sites for enzymes XbaI, Muni, Sty1 and SplI are present. The MunI and StyI sites are within the coding region of the OmpA signal sequence and are intended as the 5′ cloning sites for insertion of genes. The two oligonucleotides which make up this cassette are annealed together by mixing at a concentration of 5 pmoles/μl and heating in a waterbath, then slowly cooling to room temperature. The annealed sequence is then ligated into the SalI/EcoRI cut pTTQ9. The resulting plasmid intermediate, termed pTQOmp, is verified by DNA sequencing.

Plasmid pTTO-1 is constructed by ligating one DNA fragment from plasmid pACYC184 to two fragments generated from pTQOmp. Plasmid pACYC184 can be obtained from New England Biolabs, and a restriction map is shown in FIG. 11. An aliquot (2 μg) is digested to completion with restriction enzyme Sty1, then treated with Mung Bean Nuclease; this treatment creates blunt ends by cutting back 5′ base overhangs. Following phenol extraction and ethanol precipitation, the DNA is restricted with enzyme PvuII, generating fragments of 2348, 1081, 412 and 403 bp. The 2348 bp fragment is purified after agarose gel electrophoresis. This fragment encodes the tetracycline resistance marker and the p15A origin of replication. The fragment is then treated with calf intestinal alkaline phosphatase to remove 5′ terminal phosphates, thereby preventing the self-ligation of this molecule.

An aliquot (2 μg) of plasmid pTQOmp is then digested with enzymes SspI and EcoRI, and the 2350 bp fragment purified from unwanted fragments of 2040 bp and 170 bp following agarose gel electrophoresis; this fragment encodes the transcriptional terminator region and the laciq gene. Another aliquot (2 μg) of pTQOmp is digested with EcoRI and XmnI, generating fragments of 2289, 1670, 350 and 250 bp. The 350 bp fragment, encoding the tac promoter, OmpA signal sequence and multicloning site, is gel purified.

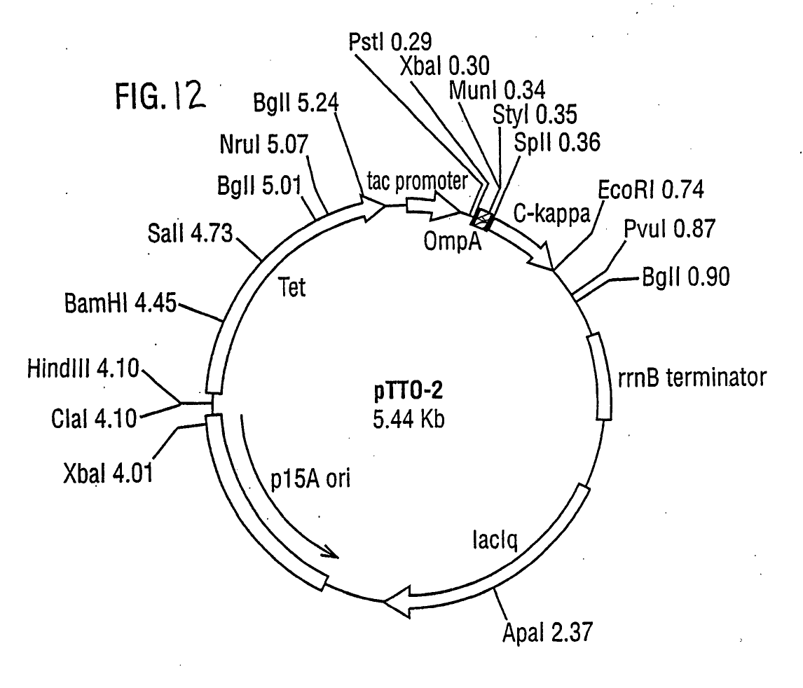

The three fragments are then ligated, using approximately equimolar amounts of each fragment, to generate the plasmid pTTO-1. All cloning junctions were verified by DNA sequencing. The restriction map of this plasmid is shown in FIG. 8. Plasmid pTTO-2 is then created by insertion of DNA encoding the human Ig light chain kappa constant domain. This was obtained as a SplI-EcoRI restriction fragment from plasmid pHC132, and inserted into the corresponding sites in pTTO-1. Plasmid pTTO-2 is shown in FIG. 12.

Humanized hTNF40 variable regions can then be inserted into pTTO-2. The variable light chain region hTNF40 μl (SEQ ID NO: 8) is obtained by PCR ‘rescue’ from the corresponding vector for mammalian cell expression pMR10.1. The OmpA leader sequence replaces the native Ig leader. The sequence of the PCR primers is shown below:

| | |
|---|---|
| 5′ primer: | |
| CGCGCGGCAATTGCAGTGGCCTTGGCTGGCTGGTTTCGCTACCGTAG | (SEQ ID NO: 79) |
| CGCAAGCTGACATTCAAATGACCCAGAGCCC | |
| 3′ primer: | |
| TTCAACTGCTCATCAGATGG | (SEQ ID NO: 80) |

Following PCR under standard conditions, the product is purified, digested with enzymes Muni and SplI then gel purified. The purified fragment is inserted into the MunI/SplI sites of pTTO-2 to create the light chain intermediate pTTO (hTNF40L).

The variable heavy chain region of gh3hTNF40.4 is shown in FIG. 15 (SEQ ID NO: 11) and is obtained in the same way from the vector pGamma-4. The sequence of the PCR primers is shown below:

| | |
|---|---|
| 5′ primer: | |
| GCTATCGCAATTGCAGTGGCGCTAGCTGGTTTCGCCACCGTGGCGCAAGCTG | (SEQ ID NO: 87) |
| AGGTTCAGCTGGTCGAGTCAGGAGGC | |
| 3′ primer: | |
| GCCTGAGTTCCACGACAC | (SEQ ID NO: 82) |

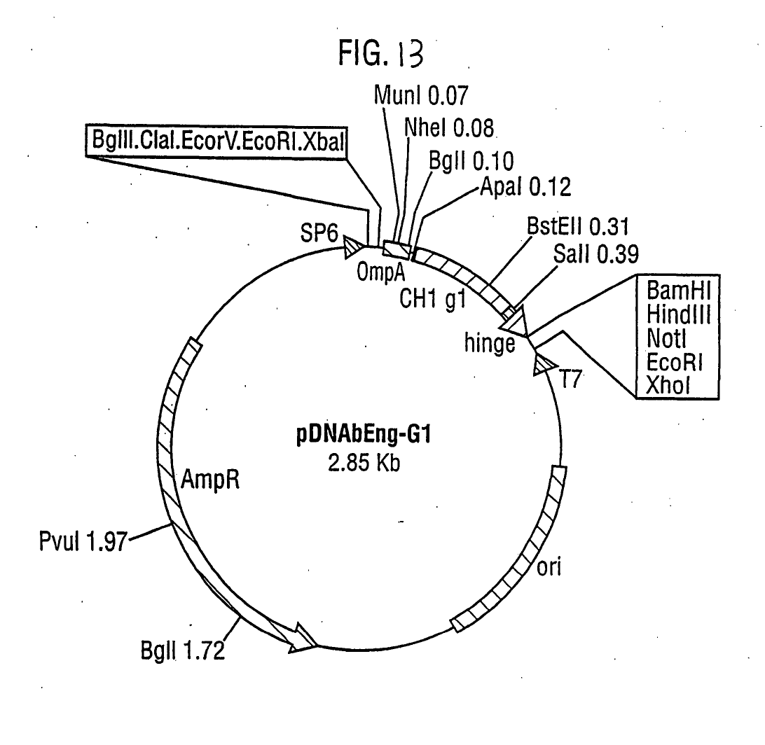

Following PCR the product is purified, digested with enzymes Nhe1 and Apa1 then sub-cloned into the vector pDNAbEng-G1 (FIG. 13). After verification by DNA sequencing, the heavy chain is restricted with enzyme EcoRI and sub-cloned into the EcoRi site of pTTO (hTNF40L) to create the E. coli expression plasmid pTTO(hTNF40).

Intergenic sequence for modified Fab expression can be optimized. In the pTTO vector, modified Fab expression occurs from a dicistronic message encoding first light chain then heavy chain. The DNA sequence between the two genes (intergenic sequence, IGS) can influence the level of expression of the heavy chain by affecting the rate of translational initiation. For example, a short intergenic sequence may result in translational coupling between the light and heavy chains, in that the translating ribosome may not fully dissociate from the mRNA after completing light chain synthesis before initiating heavy chain synthesis. The strength of any Shine Dalgarno (SD) ribosome binding site (homology to 16S rRNA) can also have an effect, as can the distance and sequence composition between the SD and the ATG start codon. The potential secondary structure of mRNA around the ATG is another important factor; the ATG should be in a ‘loop’ and not constrained within a ‘stem’, while the reverse applies to the SD. Thus by modifying the composition and length of the IGS it is possible to modify the strength of translational initiation and therefore the level of heavy chain production. It is likely that an optimum rate of translational initiation needs to be achieved to maximize expression of the heavy chain of a given modified Fab.

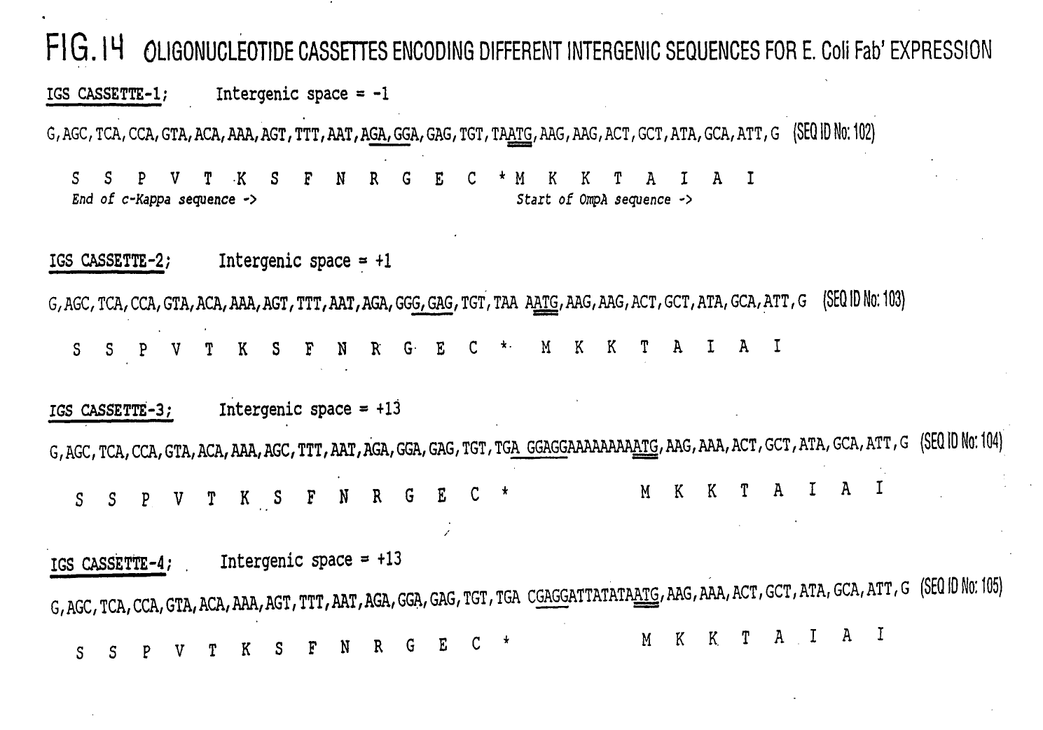

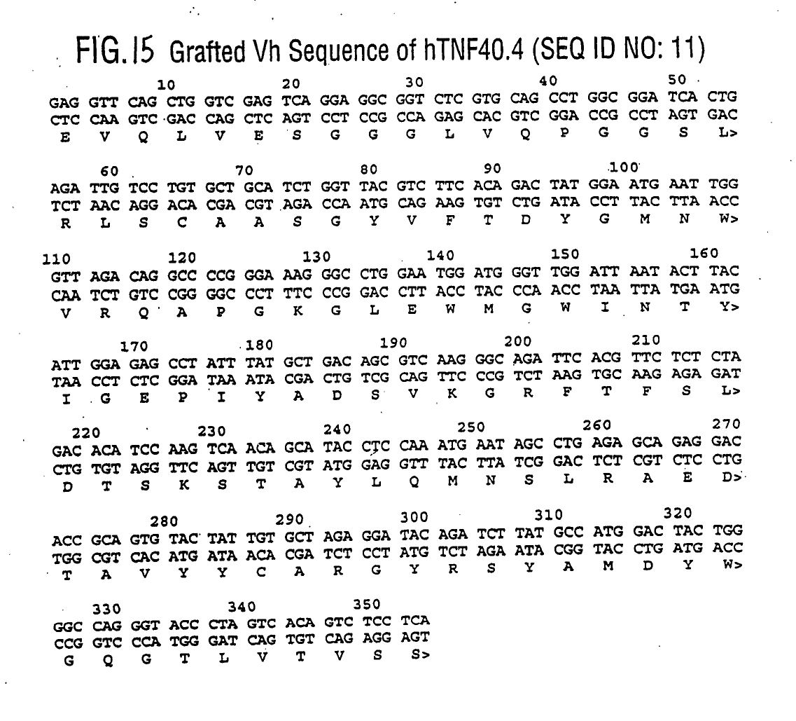

For example, with one modified Fab, a high level of expression may be tolerated, but for a different modified Fab with different amino acid sequence, a high level of expression might prove toxic, perhaps because of different efficiencies of secretion or folding. For this reason, a series of four intergenic sequences can be designed (FIG. 14), permitting the empirical determination of the optimum IGS for the hTNF40-based modified Fab. IGS1 and IGS2 have very short intergenic sequences (−1 and +1 respectively) and might be expected to give closely coupled translation; the SD sequences (underlined) are subtly different. These two sequences will most likely confer a high level of translational initiation. IGS3 and IGS4 have a longer distance between start and stop codons (+13) and differ in their sequence composition; IGS3 has a stronger SD sequence. All sequences can be studied for secondary structure and optimized as far as possible; however, with tight coupling of translation of the two chains, the lack of ribosomal dissociation means that the mRNA may not be ‘naked’ preventing secondary structure formation.

Cloning of IGS variants is performed using the IGS cassettes shown in FIG. 14 having flanking SacI and MunI cloning sites. They are built by annealing complementary oligonucleotide pairs. A vector fragment is prepared by digesting pTTO (hTNF40) with SacI and NotI, and a heavy chain fragment is prepared by digesting pDNAbEngG1 (hTNF40H) with Muni and NotI. Three-way ligations are then performed, using equimolar amounts of the two restriction fragments and approximately 0.05 pmoles of each annealed oligo cassette. This creates four expression plasmids pTTO(hTNF40 IGS-1), pTTO(hTNF40 IGS-2), pTTO(hTNF40 IGS-3), pTTO(hTNF40 IGS-4).

The four plasmids are transformed into E. coli strain W3110, along with the original expression construct, and then analyzed for expression in shake flasks as described. Based on a comparison of productivity in the fermenter the IGS construct that is the highest yielding can be selected. PEGylation of the CDR-Grafted, hTNF40-based modified Fab is then performed using a branched molecule of PEG. This is achieved by activation of a single cysteine residue in a truncated hinge region of the modified Fab, followed by reaction with (PEG)-lysyl maleimide as previously described (A. P. Chapman et al., Nature Biotechnology 17, 780-783, 1999). The PEGylated molecule, as shown in FIG. 6, is compound CDP870.

The present invention also provides a therapeutic composition comprising an anti-TNFα antibody of the present invention in combination with at least one DMARD. Furthermore, the present invention provides a method for administering the therapeutic composition to a human or animal prophylactically or therapeutically.

Suitable DMARDs for use in connection the present invention include, but are not limited to, methotrexate, sulfasalazine, leflunomide, azathioprine, cyclosporine, hydroxycloroquine, and D-pencillamine. It is known to one skilled in the art which DMARD should be used to further reduce the inflammatory response of the disease and/or to reduce any potential immunosuppressive response generated by the antibody. For example, DMARDs are known to play a potential role in the treatment of rheumatoid arthritis (RA). DMARDs as a group share common characteristics of relieving symptoms and helping to control RA by modifying its actual disease process. However, a DMARD may also be used to reduce any potential immunoresponse to the antibody Fab′ fragment CDP870. Moreover, the use of a DMARD in connection with the antibody Fab′ fragment may reduce the inflammatory response of the disease and reduce an immunosuppressive response.

### Methotrexate

Methotrexate, having the chemical formula C₂₀H₂₂N₈O₅, is an immunosuppressive agent that was first clinically used in 1947 to treat childhood leukemia and has received FDA approval as a treatment for cancer, psoriasis and rheumatoid arthritis. In the subject of the present invention, methotrexate is used in combination with the anti-TNFα antibody CDP870 as a treatment for inflammatory diseases or disorders including but not limited to rheumatoid arthritis and Crohn's Disease. Injectable or tablet formulations may be used during administration in the present invention. Methotrexate prodrugs, homologs and/or analogs (e.g. folate antagonists) are suitable and may be used in the methods and therapeutic compositions of the present invention.

### Sulfasalazine

The DMARD Sulfasalazine (Azulfidine®, Pharmacia, Peapack, N.J.) is an anti-inflammatory agent that was first introduced into clinical medicine in the late 1920's for the treatment of rheumatoid arthritis. Sulfasalazine, a combination of sulfapyridine and an aspirin-like compound, 5-aminosalicylic acid (5-ASA), has the chemical formula C₁₈H₁₄N₄O₅S and can be administered in tablet form in the methods and therapeutic compositions of the present invention.

### Leflunomide

Leflunomide is another DMARD that is used in the treatment of RA. Regular blood tests including liver function tests and blood counts are required to monitor patients using leflunomide. With side effects including gastrointestinal symptoms, skin rashes, and reversible hair loss, leflunomide should not be taken by people with active infections, or who are pregnant or nursing. Because studies have shown that leflunomide can cause birth defects in animals, women of childbearing age must take exceptional care to prevent pregnancy while taking the drug. Men planning to father a child must also make sure the medication is clear from their body when attempting to conceive.

### Azathioprine

Azathioprine is an immunosuppressant, meaning it can prevent or hinder the ability of the immune system to respond. Patients using azathioprine must be closely monitored by their doctor for the drug's potential effects on their bone marrow and liver. Other side effects to consider include an increased risk of infection.

### Cyclosporine

Cyclosporine is another immunosuppressive drug that is prescribed in combination with methotrexate for treating active, severe RA. Due to cyclosporine's potential to cause kidney damage, doctors prescribing the drug will closely monitor patients' blood pressure, kidney function, and ongoing blood tests.

### Hydroxychloroquine

Hydroxychloroquine was originally a drug for malaria that has been used to treat RA due to its ability to relieve swelling, inflammation and pain. Although hydroxychloroquine is the safest of the DMARDs, patients taking the drug require regular eye exams to check for retinal damage that hydroxychloroquine can cause on some rare occasions.

### D-Pencillamine

D-pencillamine is a DMARD that has seen its use diminish in recent years due to its significant side effects and slow action. D-pencillamine is taken as a pill on an empty stomach, and its usage also requires close supervision by a physician.

### Administration

In a preferred embodiment, the therapeutic composition comprises the anti-TNFα antibody CDP870 and the DMARD comprises at least one of methotrexate or sulfasalazine. CDP870 can be administered prior to, simultaneously with (in the same or different composition) or sequentially with the administration of the DMARD. The DMARD is administered with CDP870 to suppress any immunological side effects CDP870 may generate during its use. For example, CDP870 can be administered as adjunctive and/or concomitant therapy to methotrexate therapy. Also, alternatively, the DMARD may be conjugated to CDP870.

The therapeutic composition of the present invention may be utilized in any therapy where it is desired to reduce the level of biologically active TNFα present in the human or animal body. The TNFα may be circulating in the body or be present in an undesirably high level localized at a particular site in the body. The therapeutic composition of the present invention is preferably used for the treatment of rheumatoid arthritis, Crohn's Disease or other inflammatory disease.

Most commonly employed dosing regimens are likely to be parental administration of CDP870, once every four weeks plus oral administration of methotrexate once a week and/or the sulfasalazine either once daily or in individual doses. The therapeutic composition may be administered to a subject in a variety of ways. Preferred routes for administration include forms suitable for parenteral administration such as injection or infusion (e.g. bolus injection or continuous infusion), intravenous, subcutaneous or intramuscular administration. Where the therapeutic composition is for parenteral administration, it may take the form of a suspension, solution, emulsion in an oily or aqueous vehicle, or lyophilized powder in association with a pharmaceutically acceptable parenteral vehicle (e.g. water, saline, dextrose solution, 5% human serum albumin), and it may contain formulatory agents such as additives to maintain isotonicity (e.g. mannitol) and chemical stability (e.g. buffers), or other suspending, preservative, stabilizing and/or dispersing agents. Alternatively, solid forms suitable for solution in, or suspension in, liquid vehicles prior to administration may also be used. For example, CDP870 may be in a dry form ready for reconstitution before use. The therapeutic composition when formulated can be sterilized by commonly used techniques.

Other routes of administration for the therapeutic composition of the present invention include, but are not limited, to: oral, intra-arterial, intradermal, intramedullary, intrathecal, intraventricular, transdermal, transcutaneous (as described in W/O 98/20734), intraperitoneal, intranasal, enteral, topical, sublingual, intravaginal or rectal routes. Hyposprays may also be used to administer the therapeutic composition of the present invention.

Furthermore, the therapeutic composition may also contain a pharmaceutically acceptable carrier. A thorough discussion of pharmaceutically acceptable carriers is available in Remington's Pharmaceutical Sciences (Mack Publishing Company, N.J. 1991), of which the entire contents are incorporated herein by reference.

The pharmaceutically acceptable carrier should not itself induce the production of antibodies harmful to the subject receiving the composition and should not be toxic. Suitable carriers may include large, slowly metabolized macromolecules such as proteins, polypeptides, liposomes, polysaccharides, polylactic acids, polyglycolic acids, polymeric amino acids, amino acid copolymers and inactive virus particles. Pharmaceutically acceptable salts can also be used if desired, for example mineral acid salts, such as hydrochlorides, hydrobromides, phosphates and sulphates, or salts of organic acids, such as acetates, propionates, malonates and benzoates.

Additionally, pharmaceutically acceptable carriers of the present invention can contain liquids such as water, saline, glycerol and ethanol. Additionally, auxiliary substances, such as wetting or emulsifying agents or pH buffering substances, may be present in the therapeutic composition. Such carriers enable the therapeutic composition to be formulated as tablets, pills, capsules, liquids, gels, syrups, slurries and suspensions for ingestion by the subject. In addition, sweetening, flavoring and/or coloring agents can also be added.

Because one of the active ingredients in the therapeutic composition will be an antibody molecule, it will be susceptible to degradation in the gastrointestinal tract. Thus, if the therapeutic composition is to be administered by a route using the gastrointestinal tract, the composition will need to contain agents that protect the antibody from degradation but will release the antibody once it has been absorbed from the gastrointestinal tract.

Once formulated, the therapeutic composition of the present invention can be administered directly to the subject. The subjects to be treated can be animals. However, it is preferred that the compositions are adapted for administration to humans. As noted above, direct delivery of the compositions will generally be accomplished by injection, subcutaneously, intraperitoneally, intravenously or intramuscularly, or delivered to the interstitial space of a tissue. Dosage treatment may be a single dose schedule or a multiple dose schedule.

The therapeutic composition of the present invention is administered in therapeutically effective amounts. The term “therapeutically effective amount” refers to an amount of a therapeutic agent needed to treat, ameliorate or prevent a targeted disease or condition, or to exhibit a detectable therapeutic or preventative effect. For TNFα, a therapeutically effective amount is such that administration of the therapeutic composition results in inhibition of the biological activity of TNFα relative to the biological activity of TNFα when therapeutically effective amounts of CDP870, methotrexate, sulfasalazine or other immunosuppressive agents are not administered. A therapeutically effective amount is therefore preferably an amount of CDP870, methotrexate, sulfasalazine or other immunosuppressive agent necessary to significantly reduce or eliminate signs and symptoms associated with rheumatoid arthritis or other rheumatic-related disease. A therapeutic effective amount as used herein is not necessarily an amount such that administration of CDP870, or administration of methotrexate and/or sulfasalazine alone, must necessarily result in inhibition of the biological activity of TNFα.

For any antibody, the therapeutic effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The therapeutic effective amount for a human will depend upon the severity of the disease state, the general health, age, weight and gender of the human, diet, time and frequency of administration, drug combination(s), reaction sensitivities and tolerance/response to therapy. This amount can be determined by routine experimentation and is within the judgment of the clinician. Therefore, in a particular embodiment, CDP870 is administered in multiple doses and methotrexate or sulfasalazine is administered in low doses in a series separated by days or weeks.

Generally, for humans, a therapeutic effective amount of CDP870 will be from 200 to 800 mg once every four weeks, preferably 400 mg once every four weeks; a therapeutic effective amount of methotrexate will be from 2.5 to 50 mg per week, preferably 7.5 to 15 mg per week; and, a therapeutic effective amount for sulfasalazine will be from 0.5 to 3 g daily in divided doses, preferably 2 to 3 g daily in divided doses. This therapeutic effective amount will vary should methotrexate and sulfasalazine be combined to form the DMARD that is administered with CDP870.

As noted above, CDP870 and at least one of the DMARDs methotrexate or sulfasalazine can each be administered in single or multiple doses depending on factors such as extent of symptoms, kind of concurrent treatment and the effect desired. Also, other therapeutic regimens or agents (e.g. multiple drug regimens) can be used in combination with the therapeutic composition of the present invention. Adjustment and manipulation of established dosage ranges are well within the ability of those skilled in the art.

Once a therapeutically effective amount has been administered, a maintenance amount of CDP870, methotrexate, sulfasalazine, alone or in combination with each other can be administered in the form of a single dose, or a series of doses separated by intervals of days or weeks, to the subject. A maintenance amount is the amount necessary to maintain the reduction or elimination of the signs and symptoms associated with rheumatoid arthritis or other rheumatic disease achieved by the therapeutically effective dose. Most commonly employed treatments will be: 1) parental administration of CDP870 once every four weeks plus oral administration of methotrexate once a week, or 2) parental administration of CDP870 once every four weeks plus oral daily administration of sulfasalazine. Alternatively, a co-administration approach may be employed using parental administration of two agents such as CDP870 and methotrexate.

In one embodiment, the daily dosage of active ingredient (i.e. CDP870 and/or DMARD) can be about 0.01 to 100 mg/kg of body weight. Ordinarily, 1 to 40 mg/kg per day given in divided doses 1 to 6 times a day or in sustained release form is effective to obtain desired results. Dosage forms of the therapeutic composition suitable for internal administration generally contain from about 0.1 mg to about 500 mg of active ingredient per unit. In this therapeutic composition the active ingredient will ordinarily be present in an amount of abut 0.5-95% by weight based on the total weight of the composition.

Second or subsequent administrations can be administered at a dosage which is the same, less than or greater than the initial or previous dose administered to the individual. A second or subsequent administration is preferably during or immediately prior to relapse or a flare-up of the disease or symptoms of the disease. “Flare-up” or “relapse” includes the reappearance of one or more symptoms of rheumatoid arthritis or other rheumatic-related disease. For example, second and subsequent administrations can be given between about one day to 30 weeks from the previous administration when the subject exhibits swollen joints, morning stiffness or joint tenderness. Two, three, four or more total administrations can be delivered to the subject as needed.

It is also envisaged that the anti-TNFα antibody of the present invention will be administered by use of gene therapy. In order to achieve this, DNA sequences encoding the heavy and light chains of the anti-TNFα antibody molecule under the control of appropriate DNA components are introduced into a patient such that the antibody chains are expressed from the DNA sequences and assembled in situ. (Maulik and Patel, Molecular Biotechnology, Wiley-Liss: New York, 1997).

In a specific embodiment, the expression of CDP870 in a cell is achieved by the introduction of DNA including at least a regulatory sequence and CDP870 exons (coding regions) and may contain other sequences such as splice donor site DNA for into the genome at a specific or random site The regulatory region may encode constitutive, inducible, or tissue-specific promoters. (Birren et al., Genome Analysis A Laboratory Manual Volume 3 Cloning Systems, Cold Spring Harbor Laboratory Press: USA, 1999).

These components are introduced into the genomic DNA in a manner that results in the production and secretion of biologically active CDP-870. CDP-870 gene therapy may employ, but is not limited to, potential vehicles such as normal human cells isolated from individuals, viral-based vector systems, synthetic non-viral delivery systems such as cationic lipids or naked DNA. Alternatively, recombinant bacteria, such as E. coli expressing CDP870, may be introduced into the intestinal track of individuals such as Crohn's patients for therapeutic treatment of disease.

In an exemplary method, recombinant human or non-human animal cells may be directly implanted into patients or may be encapsulated to permit release of CDP870 but prevent the destruction of the cells by the patient's immune system.

In another exemplary method, viral vectors that contain the nucleic acid sequences corresponding to CDP870 may be used. For example, retroviral, adenoviral or adeno-associated viral vector containing components necessary for packaging of recombinant viral genome and integration into the host cell DNA may be employed.

### EXAMPLES

### Efficacy of CDP-870 in Treating the Signs and Symptoms of RA

The efficacy of CDP-870 in treating the signs and symptoms of RA by IV and SC routes of administration was examined in the Celltech-002 and -004 studies, respectively.

In the Celltech-002 study, the effects of CDP-870 were investigated in patients with active RA who had failed at least one disease modifying anti-rheumatic drug (DMARD). DMARD therapy was discontinued one month prior to study start; concomitant nonsteroidal anti-inflammatory drug (NSAID) and corticosteroid use was allowed provided the doses had been stabilized prior to entry into the study. Using a sequential ascending dose group design, three groups of patients (n=12/group) were randomized to receive in double-blinded fashion a single peripheral IV infusion of CDP-870 (n=8) or placebo (n=4) over 60 minutes. The dosage levels of CDP-870 examined were 1, 5 and 20 mg/kg. Patients were assessed over an 8-week period.

Following completion of the double-blind period, all patients were allowed the option of an open label IV infusion of CDP-870 at a dose of 1, 5 or 20 mg/kg. Patients were assessed for an additional 8-week period.

The Celltech-004 study also examined the effects of CDP-870 in patients with active RA who had failed at least one DMARD. Again, DMARD therapy was discontinued one month prior to study start; concomitant NSAID and corticosteroid use was allowed provided the doses had been stabilized prior to entry into the study. In this parallel group, two-panel study, patients were randomized to receive placebo or CDP-870 50, 100, 200 or 400 mg SC every 4 weeks (Panel 1), or placebo or CDP-870 600 or 800 mg (Panel 2) SC every 4 weeks for 12 weeks.

Following completion of the double-blind period, all patients were allowed the option of receiving open label CDP-870 200 mg SC every 4 weeks for an additional 48 weeks. The protocol was subsequently amended based on the results of the double-blind portion of Panel 1, to increase the dose to 400 mg SC every 4 weeks for an additional 108 weeks.

The overall effect of CDP870 in reducing the signs and symptoms of RA was summarized using the following global index:
American College of Rheumatology (ACR) Core Set of Disease Activity Measures for Rheumatoid Arthritis Clinical Trials. [1] A patient was considered an ACR 20/50/70 responder if 20/50/70% or greater improvement in number of swollen joints and tender joints and a 20/50/70% or greater improvement in three of the five remaining ACR core set measures (Patient's Assessment of Pain by VAS, Patient's Global Assessment of Disease Activity, Physician's Global Assessment of Disease Activity, Patient's Assessment of Physical Function by mHAQ, acute phase reactant value) was observed; and

ACR responder rates from the Celltech-002 study are shown in Table 1.

TABLE 1
ACR Responder Rates (% of Patients) at Week 8 (Celltech -002 Study)

| CDP-870 mg/kg IV | ACR20 | ACR50 |
|---|---|---|
| 0 (n = 12) | 17 | 0 |
| 1 (n-8) | 25 | 13 |
| 5 (n = 8) | 75 | 13 |
| 20 (n = 8) | 75 | 50 |

The data from this well-designed, albeit small, study show that CDP-870 given as a single IV dose of 1 mg/kg, 5 mg/kg, or 20 mg/kg is efficacious in reducing the signs and symptoms of RA over the course of 8 weeks.

The percentages of patients achieving ACR 20/50/70 response rates were also calculated for the Celltech-004 study and the data are shown in Table 2.

TABLE 2
ACR Responder Rates (% of Patients) at Week 12 (Celltech -004 Study)

| | 0-Panel 1 | 0-Panel 2 | 50 | 100 | 200 | 400 | 600 | 800 |
|---|---|---|---|---|---|---|---|---|
| | (n = 40) | (N = 44) | (n = 39) | (n = 40) | (n = 41) | (n = 42) | (n = 39) | (n = 38) |
| ACR-20 | 15 | 19 | 21 | 20 | 34 | 60* | 64* | 79* |
| ACR-50 | 0 | 7 | 8 | 5 | 17* | 40* | 33* | 47* |
| ACR-70 | 0 | 0 | 5 | 3 | 7 | 29* | 21* | 21* |

*p ≦ 0.05 versus placebo within each panel.

These composite measure data as well as the individual disease measure data (e.g., swollen joint count, tender joint count) suggest that CDP-870 at doses ≧400 mg SC every 4 weeks is efficacious in treating the signs and symptoms of RA.

The PK of CDP-870 was also evaluated in the PK interaction study with methotrexate (MTX) in RA patients (PHA-001 study). This was an open-label, nonrandomized, multi-center, add-on design study in 16 male and female patients who had a verified diagnosis of RA for a minimum of 6 months and who had been stabilized on single weekly doses of MTX (5-17.5 mg/week orally [PO]) for a minimum of 3 months. A single dose of 400 mg of CDP-870 was given by subcutaneous injection concurrently with MTX and plasma concentrations of both drugs were followed for 55 days thereafter.

PK data for CDP-870 from this study are presented in Table 3. The overall plasma exposure (AUC) to CDP-870 over the 55-day monitoring period following a single 400 mg subcutaneous dose was approximately 22000 hr*μg/mL, and Cₘₐₓ was 46.55 μg/mL. The mean Tₘₐₓ was approximately 132 hrs after dosing. The mean t½ and plasma clearance of CDP-870 were 10.7 days and 0.021 L/hr, respectively.

TABLE 3
CDP-870 Pharmacokinetic Parameters in Patients with RA Taking
Methotrexate [Arithmetic Mean (% CV; PHA-001 Study)]

Methotrexate 5-17.5
mg Weekly +
CDP-870 400 mg
SD (N = 16)

| Pharmacokinetic Parameter | Mean | (% CV) |
|---|---|---|
| AUC(0-55 days) (hr * μg/mL) | 21187.32 | (32%) |
| AUC(0-lqc) (hr * μg/mL) | 21183.68 | (32%) |
| AUC(0-∞) (hr * μg/mL) | 22419.01 | (33%) |
| Cₘₐₓ (μg/mL) | 46.55 | (39%) |
| Tₘₐₓ (hr) | 131.99 | (48%) |
| t½ (hr) | 257.71 | (42%) |
| CL/F (L/hr) | 0.021 | (50%) |

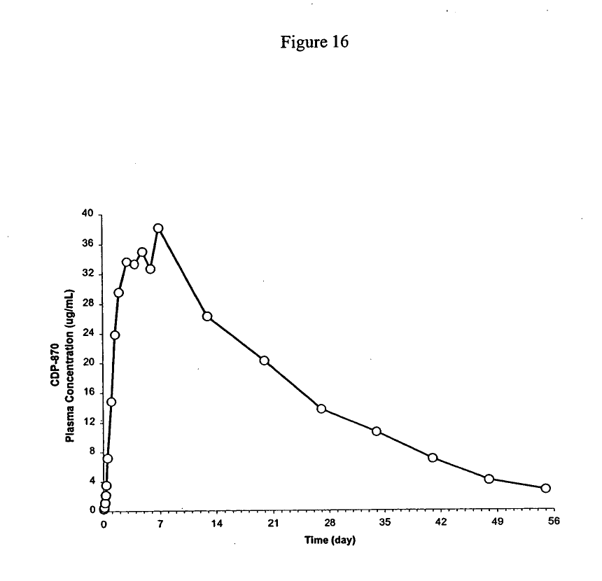

The mean plasma concentrations of CDP-870 during the 55 days after the administration of a single 400 mg dose are depicted in FIG. 16. RA patients received chronic weekly oral MTX throughout this period. The mean peak CDP-870 plasma concentration was 7 days postdose; thereafter, mean CDP-870 plasma concentrations gradually decreased. At 55 days postdose, CDP-870 concentrations were still detectable in plasma (mean of 2.74 μg/mL).

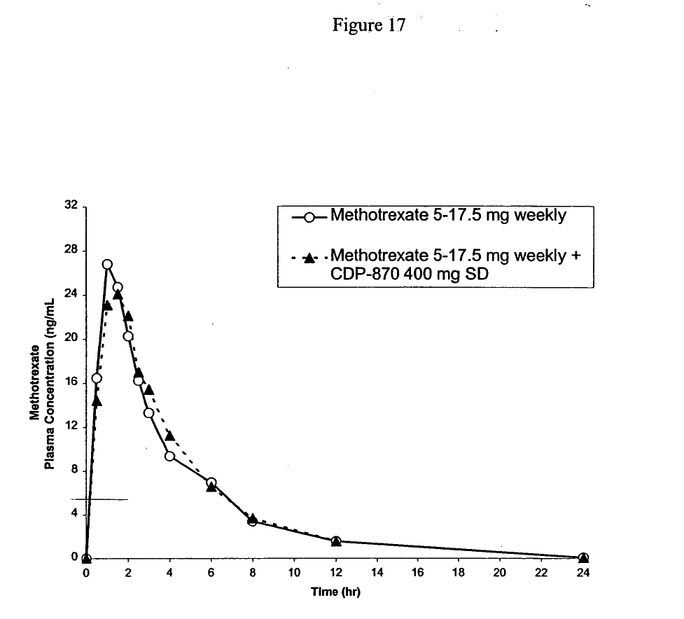

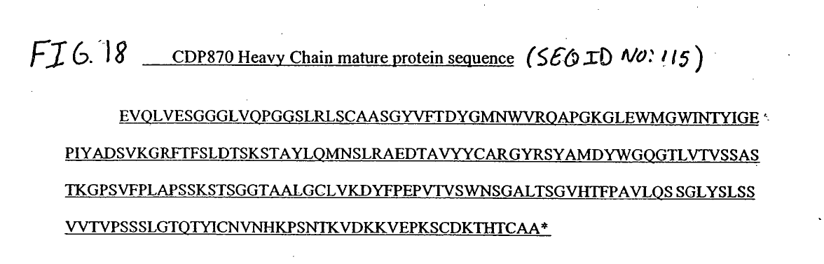

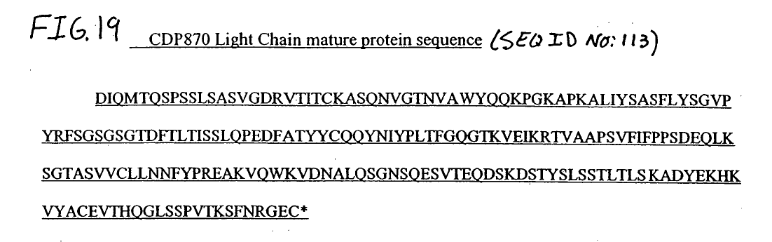

As shown in FIG. 17 the mean dose-adjusted MTX plasma concentrations following administration of MTX alone were not significantly different than those observed after coadministration of MTX with a single dose (SD) of CDP-870.

In conclusion, this study has confirmed that concurrent administration of a single 400 mg SC dose of CDP-870 with weekly individualized oral doses of 5-17.5 mg MTX in RA patients did not have a statistically or clinically meaningful effect on the overall extent of plasma exposure (AUC) or peak plasma exposure (Cₘₐₓ) of MTX.

The complete content of all publications, patents and patent applications cited in this description are herein incorporated by reference as if each individual publication, patent or patent application were specifically and individually indicated as being incorporated by reference.

The foregoing invention has been described above in some detail by way of illustration and example for the purposes of clarity of understanding. The above examples are provided for exemplification purposes only and are not intended to limit the scope of the invention, which has been described in broad terms before the examples. It will be readily apparent to one skilled in the art in light of the teachings of this invention that changes and modifications can be made without departing from the spirit and scope of the present invention.

1. A method of treating an inflammatory disease or inflammatory disorder in a person in need thereof comprising administering an anti-tumor necrosis factor alpha antibody Fab′ fragment CDP870 and at least one disease modifying anti-rheumatic drug to the person in a therapeutically effective amount.

2. The method of claim 1 wherein the disease modifying anti-rheumatic drug is methotrexate.

3. The method of claim 1 wherein the disease modifying anti-rheumatic drug is sulfasalazine.

4. A method of treating arthritis in a person in need thereof comprising administering an anti-tumor necrosis factor alpha antibody Fab′ fragment CDP870 and at least one disease modifying anti-rheumatic drug to the person in a therapeutically effective amount.

5. The method of claim 4 wherein the disease modifying anti-rheumatic drug is methotrexate.

6. The method of claim 4 wherein the disease modifying anti-rheumatic drug is sulfasalazine.

7. A method of treating rheumatoid arthritis in a person in need thereof comprising administering an anti-tumor necrosis factor alpha antibody Fab′ fragment CDP870 and at least one disease modifying anti-rheumatic drug to the person in a therapeutically effective amount.

8. The method of claim 7 wherein the disease modifying anti-rheumatic drug is methotrexate.

9. The method of claim 7 wherein the disease modifying anti-rheumatic drug is sulfasalazine.

10. A method of treating inflammatory disease or disorder in a person in need thereof comprising co-administering an anti-tumor necrosis factor alpha antibody Fab′ fragment CDP870 and at least one disease modifying anti-rheumatic drug to the person in a therapeutically effective amount.

11. The method of claim 10 wherein the disease modifying anti-rheumatic drug is methotrexate.

12. The method of claim 10 wherein the disease modifying anti-rheumatic drug is sulfasalazine.

13. A method of treating arthritis in a person in need thereof comprising co-administering an anti-tumor necrosis factor antibody Fab′ fragment CDP870 and at least one disease modifying anti-rheumatic drug to the individual in a therapeutically effective amount.

14. The method of claim 13 wherein the disease modifying anti-rheumatic drug is methotrexate.

15. The method of claim 13 wherein the disease modifying anti-rheumatic drug is sulfasalazine.

16. A method of treating rheumatoid arthritis in a person in need thereof comprising co-administering an anti-tumor necrosis factor alpha antibody Fab′ fragment CDP870 and at least one disease modifying anti-rheumatic drug to the person in a therapeutically effective amount.

17. The method of claim 16 wherein the disease modifying anti-rheumatic drug is methotrexate.

18. The method of claim 16 wherein the disease modifying anti-rheumatic drug is sulfasalazine.

19. A method of treating an inflammatory disease or disorder in a person in need thereof comprising sequentially administering an anti-tumor necrosis factor alpha antibody and sulfasalazine to the person in a therapeutically effective amount.

20. The method of claim 19 wherein the inflammatory disease is arthritis.

21. The method of claim 19 wherein the inflammatory disease is rheumatoid arthritis.

22. A method of treating arthritis in a person in need thereof comprising sequentially administering an anti-tumor necrosis factor alpha antibody Fab′ fragment CDP870 and at least one disease modifying anti-rheumatic drug to the person in a therapeutically effective amount.

23. The method of claim 22 wherein the disease modifying anti-rheumatic drug is methotrexate.

24. The method of claim 22 wherein the disease modifying anti-rheumatic drug is sulfasalazine.

25. A method of treating rheumatoid arthritis in a person in need thereof comprising sequentially administering an anti-tumor necrosis factor alpha antibody Fab′ fragment CDP870 and at least one disease modifying anti-rheumatic drug to the person in a therapeutically effective amount.

26. The method of claim 25 wherein the disease modifying anti-rheumatic drug is methotrexate.

27. The method of claim 25 wherein the disease modifying anti-rheumatic drug is sulfasalazine.

28. A method for treatment, prevention or inhibition of inflammation or inflammation-related disorder in a person in need of such treatment, prevention or inhibition, the comprising treating the person with an anti-tumor necrosis factor alpha antibody Fab′ fragment CDP 870 and a disease modifying anti-rheumatic drug in a therapeutically effective amount.

29. The method of claim 28 wherein the disease modifying anti-rheumatic drug is methotrexate.

30. The method of claim 28 wherein the disease modifying anti-rheumatic drug is sulfasalazine.

31. A method for treatment, prevention or inhibition of rheumatory arthritis or a related disorder, or inflammation-associated with rheumatory arthritis in a person in need of such treatment, prevention or inhibition, the comprising the step of treating the person with an anti-tumor necrosis factor alpha antibody Fab′ fragment CDP 870 and a disease modifying anti-rheumatic drug in a therapeutically effective amount.

32. The method of claim 31 wherein the disease modifying anti-rheumatic drug is methotrexate.

33. The method of claim 31 wherein the disease modifying anti-rheumatic drug is sulfasalazine.

34. A composition for the treatment, prevention, or inhibition of inflammation, or inflammation-associated disorder comprising an anti-tumor necrosis factor alpha antibody Fab′ fragment CDP 870 and a disease modifying anti-rheumatic drug.

35. The composition of claim 34 wherein the disease modifying anti-rheumatic drug is methotrexate.

36. The composition of claim 34 wherein the disease modifying anti-rheumatic drug is sulfasalazine.

37. A composition for the treatment, prevention or inhibition or pain of rheumatoid arthritis or rheumatoid arthritis associated disorder comprising an anti-tumor necrosis factor alpha antibody Fab′ fragment CDP 870 and a disease modifying anti-rheumatic drug.

38. The composition of claim 37 wherein the disease modifying anti-rheumatic drug is methotrexate.

39. The composition of claim 37 wherein the disease modifying anti-rheumatic drug is sulfasalazine.

40. A kit that is suitable for use in the treatment, prevention or inhibition of pain, inflammation or inflammation-associated disorder comprising a first dosage form of an anti-tumor necrosis factor alpha antibody Fab′ fragment CDP 870 and a second dosage form of a disease modifying anti-rheumatic drug, said first dosage form and said second dosage form provided in quantities which comprise a therapeutically effect amount of the compounds for the treatment, prevention or inhibition of cardiovascular disease or disorder.

41. The kit of claim 40 wherein the disease modifying anti-rheumatic drug is methotrexate.

42. The kit of claim 42 wherein the disease modifying anti-rheumatic drug is sulfasalazine.

43. A kit that is suitable for use in the treatment, prevention or inhibition of rheumatoid arthritis or a rheumatoid arthritis-associated disorder comprising a first dosage form of an anti-tumor necrosis factor alpha antibody Fab′ fragment CDP 870 and a second dosage form of a disease modifying anti-rheumatic drug, said first dosage form and said second dosage form provided in quantities which comprise a therapeutically effect amount of the compounds for the treatment, prevention or inhibition of cardiovascular disease or disorder.

44. The kit of claim 43 wherein the disease modifying anti-rheumatic drug is methotrexate.

45. The kit of claim 43 wherein the disease modifying anti-rheumatic drug is sulfasalazine.

46. A kit that is suitable for use in the treatment, prevention or inhibition of pain, inflammation or inflammation-associated disorder comprising a dosage form of an anti-tumor necrosis factor alpha antibody Fab′ fragment CDP 870 and a disease modifying anti-rheumatic drug wherein said dosage form is provided in quantities which comprise a therapeutically effect amount of the compounds for the treatment, prevention or inhibition of cardiovascular disease or disorder.

47. The kit of claim 46 wherein the disease modifying anti-rheumatic drug is methotrexate.

48. The kit of claim 46 wherein the disease modifying anti-rheumatic drug is sulfasalazine.

49. A method of treating an inflammatory disease or inflammatory disorder in a person in need thereof comprising:
(a) preparing a therapeutic composition comprising a therapeutically effective amount of an anti-tumor necrosis factor alpha antibody Fab′ fragment CDP870 and at lease one disease modifying anti-rheumatic drug; and
(b) administering in a repetitive dosing regimen the therapeutic composition to the person.

50. The method of claim 49 wherein the repetitive dosing regimen of the anti-tumor necrosis factor alpha antibody Fab′ fragment CDP870 is about once every four weeks.

51. The method of claim 50 wherein the therapeutic effective amount of the anti-tumor necrosis factor alpha antibody Fab′ fragment CDP870 is about 200 mg to about 800 mg.

52. The method of claim 50 wherein the therapeutic effective amount of the anti-tumor necrosis factor alpha antibody Fab′ fragment CDP870 is about 400 mg.

53. The method of claim 49 wherein the disease modifying anti-rheumatic is methotrexate.

54. The method of claim 53 wherein the repetitive dosing regimen of the methotrexate is about once every week.

55. The method of claim 54 wherein the therapeutic effective amount of methotrexate is about 2.5 mg to about 50 mg.

56. The method of claim 54 wherein the therapeutic effective amount of the methotrexate is about 7.5 mg to about 15 mg.

57. The method of claim 49 wherein the disease modifying anti-rheumatic is sulfasalazine.

58. The method of claim 57 wherein the repetitive dosing regimen of the sulfasalazine is about once every day.

59. The method of claim 58 wherein the therapeutic effective amount of sulfasalazine is about 0.5 g to about 3 g.

60. The method of claim 58 wherein the therapeutic effective amount of the methotrexate is about 2 g to about 3 g.

61. A method of modulating an inflammatory disease or inflammatory disorder in a person in need thereof comprising administering an anti-tumor necrosis factor alpha antibody Fab′ fragment CDP870 and at least one disease modifying anti-rheumatic drug to the person in a therapeutically effective amount.

62. The method of claim 61 wherein the disease modifying anti-rheumatic drug is methotrexate.

63. The method of claim 61 wherein the disease modifying anti-rheumatic drug is sulfasalazine.

64. A method of modulating arthritis in a person in need thereof comprising administering an anti-tumor necrosis factor alpha antibody Fab′ fragment CDP870 and at least one disease modifying anti-rheumatic drug to the person in a therapeutically effective amount.

65. The method of claim 64 wherein the disease modifying anti-rheumatic drug is methotrexate.

66. The method of claim 64 wherein the disease modifying anti-rheumatic drug is sulfasalazine.

67. A method of modulating rheumatoid arthritis in a person in need thereof comprising administering an anti-tumor necrosis factor alpha antibody Fab′ fragment CDP870 and at least one disease modifying anti-rheumatic drug to the person in a therapeutically effective amount.

68. The method of claim 67 wherein the disease modifying anti-rheumatic drug is methotrexate.

69. The method of claim 67 wherein the disease modifying anti-rheumatic drug is sulfasalazine.

70. A method of modulating inflammatory disease or disorder in a person in need thereof comprising co-administering an anti-tumor necrosis factor alpha antibody Fab′ fragment CDP870 and at least one disease modifying anti-rheumatic drug to the person in a therapeutically effective amount.

71. The method of claim 70 wherein the disease modifying anti-rheumatic drug is methotrexate.

72. The method of claim 70 wherein the disease modifying anti-rheumatic drug is sulfasalazine.

73. A method of modulating arthritis in a person in need thereof comprising co-administering an anti-tumor necrosis factor antibody Fab′ fragment CDP870 and at least one disease modifying anti-rheumatic drug to the individual in a therapeutically effective amount.

74. The method of claim 73 wherein the disease modifying anti-rheumatic drug is methotrexate.

75. The method of claim 73 wherein the disease modifying anti-rheumatic drug is sulfasalazine.

76. A method of modulating rheumatoid arthritis in a person in need thereof comprising co-administering an anti-tumor necrosis factor alpha antibody Fab′ fragment CDP870 and at least one disease modifying anti-rheumatic drug to the person in a therapeutically effective amount.

77. The method of claim 76 wherein the disease modifying anti-rheumatic drug is methotrexate.

78. The method of claim 76 wherein the disease modifying anti-rheumatic drug is sulfasalazine.

79. A method of modulating an inflammatory disease or disorder in a person in need thereof comprising sequentially administering an anti-tumor necrosis factor alpha antibody and sulfasalazine to the person in a therapeutically effective amount.

80. The method of claim 79 wherein the inflammatory disease is arthritis.

81. The method of claim 79 wherein the inflammatory disease is rheumatoid arthritis.

82. A method of modulating arthritis in a person in need thereof comprising sequentially administering an anti-tumor necrosis factor alpha antibody Fab′ fragment CDP870 and at least one disease modifying anti-rheumatic drug to the person in a therapeutically effective amount.

83. The method of claim 82 wherein the disease modifying anti-rheumatic drug is methotrexate.

84. The method of claim 82 wherein the disease modifying anti-rheumatic drug is sulfasalazine.

85. A method of modulating rheumatoid arthritis in a person in need thereof comprising sequentially administering an anti-tumor necrosis factor alpha antibody Fab′ fragment CDP870 and at least one disease modifying anti-rheumatic drug to the person in a therapeutically effective amount.

86. The method of claim 85 wherein the disease modifying anti-rheumatic drug is methotrexate.

87. The method of claim 85 wherein the disease modifying anti-rheumatic drug is sulfasalazine.


SEQUENCE LISTING (117 sequences)

SEQ ID NO: 1 - protein, 5 aa, Artificial Sequence
  Mouse mononclonal antibody hTNF40 CDRH1
DYGMN

SEQ ID NO: 2 - protein, 17 aa, Artificial Sequence
  Mouse monoclonal antibody hTNF40/human hybrid CDRH2
WINTYIGEPIYADSVKG

SEQ ID NO: 3 - protein, 9 aa, Artificial Sequence
  Mouse monoclonal antibody hTNF40 CDRH3
GYRSYAMDY

SEQ ID NO: 4 - protein, 11 aa, Artificial Sequence
  Mouse monoclonal antibody hTNF40 CDRL1
KASQNVGTNVA

SEQ ID NO: 5 - protein, 7 aa, Artificial Sequence
  Mouse monoclonal antibody hTNF40 CDRL2
SASFLYS

SEQ ID NO: 6 - protein, 9 aa, Artificial Sequence
  Mouse monoclonal antibody hTNF40 CDRL3
QQYNIYPLT

SEQ ID NO: 7 - protein, 17 aa, Artificial Sequence
  Mouse monoclonal antibody hTNF40 CDRH2
WINTYIGEPIYVDDFKG

SEQ ID NO: 8 - DNA, 321 bp, Artificial Sequence
  CDS (1)...(321): Synthetic hTNF40-gL1
gacattcaaatgacccagagcccatccagcctgagcgcatctgtaggagaccgggtcaccatcacttgtaaagccagtcagaacgtaggtactaacgtagcctggtatcagcaaaaaccaggtaaagccccaaaagccctcatctacagtgcctctttcctctatagtggtgtaccatacaggttcagcggatccggtagtggtactgatttcaccctcacgatcagtagcctccagccagaagatttcgccacttattactgtcaacagtataacatctacccactcacattcggtcagggtactaaagtagaaatcaaa

SEQ ID NO: 9-10 - skipped (2 entries)

SEQ ID NO: 11 - DNA, 354 bp, Artificial Sequence
  CDS (1)...(354): Grafted Heavy Chain for Modified Fab
gaggttcagctggtcgagtcaggaggcggtctcgtgcagcctggcggatcactgagattgtcctgtgctgcatctggttacgtcttcacagactatggaatgaattgggttagacaggccccgggaaagggcctggaatggatgggttggattaatacttacattggagagcctatttatgctgacagcgtcaagggcagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctca

SEQ ID NO: 12-82 - skipped (71 entries)

SEQ ID NO: 83 - protein, 23 aa, Artificial Sequence
  human group 1 consensus framework L1
DIQMTQSPSSLSASVGDRVTITC

SEQ ID NO: 84 - protein, 23 aa, Artificial Sequence
  Human hTNF40 framework L1
DIVMTQSQKFMSTSVGDRVSVTC

SEQ ID NO: 85 - protein, 15 aa, Artificial Sequence
  human group 1 consensus framework L2
WYQQKPGKAPKLLIY

SEQ ID NO: 86 - protein, 15 aa, Artificial Sequence
  Human hTNF40 framework L2
WYQQKPGQSPKALIY

SEQ ID NO: 87 - protein, 32 aa, Artificial Sequence
  human group 1 consensus framework L3
GVPSRFSGSGSGTDFTLTISSLQPEDFATYYC

SEQ ID NO: 88 - protein, 32 aa, Artificial Sequence
  Human hTNF40 framework L3
GVPYRFTGSGSGTDFTLTISTVQSEDLAEYFC

SEQ ID NO: 89 - protein, 11 aa, Artificial Sequence
  human group 1 consensus framework L4
FGQGTKVEIKR

SEQ ID NO: 90 - protein, 11 aa, Artificial Sequence
  Human hTNF40 framework L4
FGAGTKLELKR

SEQ ID NO: 91 - protein, 30 aa, Artificial Sequence
  human group 1 consensus framework H1
QVQLVQSGAEVKKPGASVKVSCKASGYTFT

SEQ ID NO: 92 - protein, 30 aa, Artificial Sequence
  Human hTNF40 framework H1
QIQLVQSGPELKKPGETVKISCKASGYVFT

SEQ ID NO: 93 - protein, 14 aa, Artificial Sequence
  human group 1 consensus framework H2
WVRQAPGQGLEWMG

SEQ ID NO: 94 - protein, 14 aa, Artificial Sequence
  Human hTNF40 framework H2
WVKQAPGKAFKWMG

SEQ ID NO: 95 - protein, 32 aa, Artificial Sequence
  human group 1 consensus framework H3
RVTITRDTSTSTAYMELSSLRSEDTAVYYCAR

SEQ ID NO: 96 - protein, 32 aa, Artificial Sequence
  Human hTNF40 framework H3
RFAFSLETSASTAFLQINNLKNEDTATYFCAR

SEQ ID NO: 97 - protein, 11 aa, Artificial Sequence
  human group 1 consensus framework H4
WGQGTLVTVSS

SEQ ID NO: 98 - protein, 11 aa, Artificial Sequence
  human group 1 consensus framework H4
WGQGTLVTVSS

SEQ ID NO: 99 - DNA, 324 bp, murine
  CDS (1)...(324): mouse hTNF40 light chain variable domain
gacattgtgatgacccagtctcaaaaattcatgtccacatcagtaggagacagggtcagcgtcacctgcaaggccagtcagaatgtgggtactaatgtagcctggtatcaacagaaaccaggacaatctcctaaagcactgaattactcggcatccttcctatatagtggagtcccttatcgcttcacaggcagtggatctgggacagatttcactctcaccatcagcactgtgcagtctgaagacttggcagagtatttctgtcagcaatataacatctatcctctcacgttcggtgctgggaccaagctggagctgaaacgt

SEQ ID NO: 100 - DNA, 354 bp, murine
  CDS (1)...(354): mouse hTNF40 heavy chain variable domain
cagatccagttggtgcagtctggacctgagctgaagaagcctggagagacagtcaagatctcctgcaaggcttctggatatgttttcacagactatggaatgaattgggtgaagcaggctccaggaaaggctttcaagtggatgggctggataaacacctacattggagagccaatatatgttgatgacttcaagggacgatttgccttctctttggaaacctctgccagcactgcctttttgcagatcaacaacctcaaaaatgaggacacggctacatatttctgtgcaagaggttaccggtcctatgctatggactactggggtcaaggaacctcagtcaccgtctcttca

SEQ ID NO: 101 - DNA, 84 bp, Artificial Sequence
  CDS (29)...(67): Ompa oligonucleotide adaptor
tcgagttctagataacgaggcgtaaaaaatgaaaaagacagctatcgcaattgcagtggccttggctctgacgtacgagtcagg

SEQ ID NO: 102 - DNA, 67 bp, Artificial Sequence
  CDS (2)...(40): IGS cassette-1
  CDS (43)...(66): IGS cassette-1
gagctcaccagtaacaaaaagttttaatagaggagagtgttaatgaagaagactgctatagcaattg

SEQ ID NO: 103 - DNA, 69 bp, Artificial Sequence
  CDS (2)...(43): IGS cassette-2
  CDS (45)...(68): IGS cassette-2
gagctcaccagtaacaaaaagttttaatagaggggagtgttaaaatgaagaagactgctatagcaattg

SEQ ID NO: 104 - DNA, 81 bp, Artificial Sequence
  CDS (2)...(43): IGS cassette-3
  CDS (57)...(80): IGS cassette-3
gagctcaccagtaacaaaaagctttaatagaggagagtgttgaggaggaaaaaaaaatgaagaaaactgctatagcaattg

SEQ ID NO: 105 - DNA, 81 bp, Artificial Sequence
  CDS (2)...(43): IGS cassette-4
  CDS (57)...(80): IGS cassette-4
gagctcaccagtaacaaaaagttttaatagaggagagtgttgacgaggattatataatgaagaaaactgctatagcaattg

SEQ ID NO: 106 - protein, 30 aa, Artificial Sequence
  human group 3 consensus framework H1
EVQLVESGGGLVQPGGSLRLSCAASGFTFS

SEQ ID NO: 107 - protein, 14 aa, Artificial Sequence
  human group 3 consensus framework H2
WVRQAPGKGLEWVS

SEQ ID NO: 108 - protein, 32 aa, Artificial Sequence
  human group 3 consensus framework H3
RFTISRDNSKNTLYLQMNSLRAEDTAVYYCAR

SEQ ID NO: 109 - protein, 11 aa, Artificial Sequence
  human group 3 consensus framework H4
WGQGTLVTVSS

SEQ ID NO: 110-112 - skipped (3 entries)

SEQ ID NO: 113 - protein, 214 aa, Artificial Sequence
  Synthetic CDP870 Light chain mature protein sequence
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - skipped

SEQ ID NO: 115 - protein, 229 aa, Artificial Sequence
  Synthetic CDP870 Heavy chain mature protein sequence
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 116 - DNA, 1477 bp, Artificial Sequence
  Synthetic CDP870 nucleic acid sequence (sense strand)
atgaaaaagacagctatcgcaattgcagtggccttggctggtttcgctaccgtagcgcaagctgacattcaaatgacccagagcccatccagcctgagcgcatctgtaggagaccgggtcaccatcacttgtaaagccagtcagaacgtaggtactaacgtagcctggtatcagcaaaaaccaggtaaagccccaaaagccctcatctacagtgcctctttcctctatagtggtgtaccatacaggttcagcggatccggtagtggtactgatttcaccctcacgactagtagcctccagccagaagatttcgccacttattactgtcaacagtataacatctacccactcacattcggtcagggtactaaagtagaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgaggaaagcagactacgagaaacacaaagcttacgcctgcgaagtcacccatcagggcctgagctcaccagtaacaaaaagctttaatagaggagagtgttgaggaggaaaaaaaaatgaagaaaactgctatagcaattgcagtggcgctagctggtttcgccaccgtggcgcaagctgaggttcagctggtcgagtcaggaggcggtctcgtgcagcctggcggatcactgagattgtcctgtgctgcatctggttacgtcttcacagactatggaatgaattgggttagacaggccccgggaaagggcctcgaatggatgggttggattaatacttacattggagagcctatttatgctgacagcgtcaaggcgagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtccacaagaaagttgagcccaaatcttgtgacaaaactcacacatgcgccgcgtgatga

SEQ ID NO: 117 - DNA, 1477 bp, Artificial Sequence
  Synthetic CDP870 nucleic acid sequence (anti-sense strand)
tcatcacgcggcgcatgtgtgagttttgtcacaagatttgggctcaactttcttgtggaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaagctgaggagactgtgactagggtaccctggccccagtagtccatggcataagatctgtatcctctagcacaatagtacactgcggtgtcctctgctctcaggctattcatttggaggtatgctgttgacttggatgtgtctagagagaacgtgaatctcgccttgacgctgtcagcataaataggctctccaatgtaagtattaatccaacccatccattcgaggccctttcccggggcctgtctaacccaattcattccatagtctgtgaagacgtaaccagatgcagcacaggacaatctcagtgatccgccaggctgcacgagaccgcctcctgactcgaccagctgaacctcagcttgcgccacggtggcgaaaccagctagcgccactgcaattgctatagcagttttcttcatttttttttcctcctcaacactctcctctattaaagctttttgttactggtgagctcaggccctgatgggtgacttcgcaggcgtaagctttgtgtttctcgtagtctgctttcctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggggccgctaccgtacgtttgatttctactttagtaccctgaccgaatgtgagtgggtagatgttatactgttgacagtaataagtggcgaaatcttctggctggaggctactagtcgtgagggtgaaatcagtaccactaccggatccgctgaacctgtatggtacaccactatagaggaaagaggcactgtagatgagggcttttggggctttacctggtttttgctgataccaggctacgttagtacctacgttctgactggctttacaagtgatggtgacccggtctcctacagatgcgctcaggctggatgggctctgggtcatttgaatgtcagcttgcgctacggtagcgaaaccagccaaggccactgcaattgcgatagctgtctttttcat